SEQUENCE LISTING (9 sequences)

SEQ ID NO: 1 - DNA, 786 bp, Artificial Sequence
  construct encoding truncated, soluble human DRA1*0101 fused to linker and leucine zipper motif
atggccataagtggagtccctgtgctaggatttttcatcatagctgtgctgatgagcgctcaggaatcatgggctdraatcaaagaagaacatgtgatcatccaggccgagttctatctgaatcctgaccaatcaggcgagtttatgtttgacdratttgatggtgatgagattttccatgtggatatggcaaagaaggagacggtctggcggcttgaagaatttggacgadratttgccagctttgaggctcaaggtgcattggccaacatagctgtggacaaagccaacctggaaatcatgacaaagdracgctccaactatactccgatcaccaatgtacctccagaggtaactgtgctcacgaacagccctgtggaactgagadragagcccaacgtcctcatctgtttcatcgacaagttcaccccaccagtggtcaatgtcacgtggcttcgaaatggadraaaacctgtcaccacaggagtgtcagagacagtcttcctgcccagggaagaccaccttttccgcaagttccactatdractccccttcctgccctcaactgaggacgtttacgactgcagggtggagcactggggcttggatgagcctcttctcdraaagcactgggagtttgatgctccaagccctctcccagagactacagaggtcgacggaggaggaggtggagctcagctcgaaaaagagctccaggccctggagaaggaaaatgcacagctggaatgggagttgcaagcactggaaaaggaactggctcagtga

SEQ ID NO: 2 - protein, 253 aa, Artificial Sequence
  amino acid sequence encoded by SEQ ID NO1
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGAQLEKELQALEKENAQLEWELQALEKELAQ

SEQ ID NO: 3 - DNA, 822 bp, Artificial Sequence
  construct encoding truncated, soluble human DRB1*040101 fused to a linker and leucine zipper motif
atggtgtgtctgaagttccctggaggctcctgcatggcagctctgacagtgacactgatggtgctgagctccccadrbctggctttggctggggacacccgaccacgtttcttggagcaggttaaacatgagtgtcatttcttcaacgggacgdrbgagcgggtgcggttcctggacagatacttctatcaccaagaggagtacgtgcgcttcgacagcgacgtgggggagdrbtaccgggcggtgacggagctggggcggcctgatgccgagtactggaacagccagaaggacctcctggagcagaagdrbcgggccgcggtggacacctactgcagacacaactacggggttggtgagagcttcacagtgcagcggcgagtctatdrbcctgaggtgactgtgtatcctgcaaagacccagcccctgcagcaccacaacctcctggtctgctctgtgaatggtdrbttctatccaggcagcattgaagtcaggtggttccggaacggccaggaagagaagactggggtggtgtccacaggcdrbctgatccagaatggagactggaccttccagaccctggtgatgctggaaacagttcctcggagtggagaggtttacdrbacctgccaagtggagcacccaagcctgacgagccctctcacagtggaatggagagcacggtctgaatctgcacagdrbagcaaggtcgacggaggaggaggtggagctcagttgaaaaagaaattgcaagcactgaagaaaaagaacgctcagctgaagtggaaacttcaagccctcaagaagaaactcgcccagtga

SEQ ID NO: 4 - protein, 264 aa, Artificial Sequence
  amino acid sequence encoded by SEQ ID NO3
MVCLKFPGGSCMAALTVTLMVLSSPLALAGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKVDGGGGGAQLKKKLQALKKKNAQLKWKLQALKKKLAQ

SEQ ID NO: 5 - DNA, 825 bp, Artificial Sequence
  construct encoding truncated, soluble human DRB1*0103 fused to a linker and leucine zipper motif
atggtgtgtctgaagctccctggaggctcctgcatgacagcgctgacagtgacactgatggtgctgagctccccadrbctggctttggctggggacacccgaccacgtttcttgtggcagcttaagtttgaatgtcatttcttcaatgggacgdrbgagcgggtgcggttgctggaaagatgcatctataaccaagaggagtccgtgcgcttcgacagcgacgtgggggagdrbtaccgggcggtgacggagctggggcggcctgatgccgagtactggaacagccagaaggacatcctggaagacgagdrbcgggccgcggtggacacctactgcagacacaactacggggttggtgagagcttcacagtgcagcggcgagttgagdrbcctaaggtgactgtgtatccttcaaagacccagcccctgcagcaccacaacctcctggtctgctctgtgagtggtdrbttctatccaggcagcattgaagtcaggtggttccggaacggccaggaagagaaggctggggtggtgtccacaggcdrbctgatccagaatggagattggaccttccagaccctggtgatgctggaaacagttcctcggagtggagaggtttacdrbacctgccaagtggagcacccaagtgtgacgagccctctcacagtggaatggagagcacggtctgaatctgcacagdrbagcaaggtcgacggaggaggaggtggagctcagttgaaaaagaaattgcaagcactgaagaaaaagaacgctcagdrbctgaagtggaaacttcaagccctcaagaagaaactcgcccagtga

SEQ ID NO: 6 - protein, 264 aa, Artificial Sequence
  amino acid sequence encoded by SEQ ID NO5
MVCLKLPGGSCMTALTVTLMVLSSPLALAGDTRPRFLWQLKFECHFFNGTERVRLLERCIYNQEESVRFDSDVGEYRAVTELGRPDAEYWNSQKDILEDERAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKVDGGGGGAQLKKKLQALKKKNAQLKWKLQALKKKLAQ

SEQ ID NO: 7 - protein, 7 aa, Artificial Sequence
  amino acid linker sequence
DVGGGGG

SEQ ID NO: 8 - protein, 228 aa, Artificial Sequence
  sHLA-DRA1*01 01 ACIDp1
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGAQLEKELQALEKENAQLEWELQALEKELAQ

SEQ ID NO: 9 - protein, 235 aa, Artificial Sequence
  sHLA-DRB1*1101 BASEp1
GDTRPRFLEYSTSECHFFNGTERVRFLDRYFYNQEEYVRFDSDVGEFRAVTELGRPDEEYWNSQKDFLEDRRAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKVDGGGGGAQLKKKLQALKKKNAQLKWKLQALKKKLAQ

(19) United States
(12) Patent Application Publication
(10) Pub. No.: US 2013/0131573 A1
(43) Pub. Date: May 23, 2013

(54) SELECTIVE ANTI-HLA ANTIBODY REMOVAL DEVICE AND METHODS OF PRODUCTION AND USE THEREOF

(71) Applicants: William H. Hildebrand, Edmond, OK (US); Rico Buchli, Edmond, OK (US); Curtis McMurtrey, Moore, OK (US); Steven Cate, Moore, OK (US)

(72) Inventors: William H. Hildebrand, Edmond, OK (US); Rico Buchli, Edmond, OK (US); Curtis McMurtrey, Moore, OK (US); Steven Cate, Moore, OK (US)

(21) Appl. No.: 13/460,433

(22) Filed: Apr. 30, 2012

Related U.S. Application Data
(63) Continuation-in-part of application No. 12/859,002, filed on Aug. 18, 2010.
(60) Provisional application No. 61/480,865, filed on Apr. 29, 2011.
(60) Provisional application No. 61/234,937, filed on Aug. 18, 2009.
(60) Provisional application No. 61/333,827, filed on May 12, 2010.

(51) Int. Cl. A61M 1/30

(52) U.S. Cl. CPC A61M 1/30
USPC 604/6.04; 530/403

(57) ABSTRACT

An anti-MHC removal device includes a serologically active, soluble MHC moiety covalently coupled to a solid support. Methods of production include covalently coupling the serologically active, soluble MHC moiety to the solid support. Methods of use of the anti-MHC removal device include contacting a biological sample with the device so that antibodies specific for the MHC moiety are removed from the biological sample. The biological sample is then recovered.

### CROSS REFERENCE TO RELATED APPLICATIONS

This application claims benefit under 35 U.S.C. 119(e) of U.S. provisional application Ser. No. 61/480,865, filed Apr. 29, 2011.

This application is also a continuation-in-part of U.S. Ser. No. 12/859,002, filed Aug. 18, 2010; which claims benefit under 35 U.S.C. 119(e) of US provisional applications Ser. No. 61/234,937, filed Aug. 18, 2009; and Ser. No. 61/333,827, filed May 12, 2010.

The entire contents of each of the above-referenced patents and patent applications are hereby expressly incorporated herein by reference.

### STATEMENT REGARDING FEDERALLY SPONSORED RESEARCH OR DEVELOPMENT

Not Applicable.

### BACKGROUND OF THE INVENTIVE CONCEPT(S)

1. Field of the Invention

The presently disclosed and claimed inventive concept(s) relates generally to a methodology of removing anti-HLA antibodies from a sample, as well as a device utilized therefor.

2. Description of the Background Art

Human cells express on their surface an incredibly large number of membrane-bound proteins, all of which display individual properties and physiological functions. From this large array of surface cell proteins, a number of clinical procedures require characterization of the human major histocompatibility complex (MHC) class I and II membrane-bound molecules. The human MHC class I and class II molecules are known as human leukocyte antigens, or HLA. The HLA class I and class II molecules are responsible for presenting peptide antigens to receptors located on the surface of T-lymphocytes, Natural Killer Cells (NK), and possibly other immune effector and regulatory cells. Display of peptide antigens on the MHC I and MHC II molecules are the basis for the recognition of “self vs. non-self” and the onset of important immune responses such as transplant rejection, graft-versus-host-disease, autoimmune disease, and healthy anti-viral and anti-bacterial immune responses.

HLA class I and class II molecules differ from person to person. Each person expresses a different complement of class I and class II on the surface of their cells. For transplant purposes it is important to determine which of the multiple HLA expressed on a cell are recognized by the antibodies of another individual. The presence of anti-HLA antibodies in a transplant recipient can lead to hyperacute organ rejection. It is often difficult to determine which of many HLA are recognized by antibodies because sera can have antibodies to non-HLA proteins and multiple HLA molecules, and sera may crossreact among different HLA molecules. With many human proteins, many HLA proteins, antibodies to multiple human proteins, and antibodies crossreactive to various HLA proteins, it can be difficult when screening patients for organ transplantation to ascertain which of the many HLA in the population, and expressed on an organ to be transplanted, are recognized by antibodies. Antibodies to HLA proteins may also lead to problems during the transfusion of blood products, whereby antibodies in the blood of the blood donor may react with the HLA class I and class II antigens of the recipient of the blood product. Antibodies in the blood product that recognize the recipient's HLA may lead to transfusion related acute lung injury (TRAM.

Class I MHC molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptide antigens bound and presented by HLA class II are derived from extracellular foreign antigens, such as products of bacteria that multiply outside of cells, wherein such products include protein toxins secreted by the bacteria or any other bacterial protein to which the human immune system might respond in a protective manner. In this manner, class II molecules convey information regarding the existence of pathogens in extracellular spaces that are accessible to the cell displaying the class II molecule. HLA class II expressing cells then present peptide antigens derived from the extracellular antigen/bacteria to immune effector cells, including but not limited to, CD4⁺ helper T cells, thereby helping to eliminate such pathogens. The elimination of such pathogens is accomplished by both helping B cells make antibodies against microbes, as well as toxins produced by such microbes, and by activating macrophages to destroy ingested microbes.

HLA class I and class II molecules exhibit extensive polymorphism generated by systematic recombinatorial and point mutation events; as such, hundreds of different HLA types exist throughout the world's population, resulting in substantial immunologic diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, and because of their tremendous diversity, large quantities of individual HLA proteins are required in order to effectively study transplantation, autoimmunity disorders, and for vaccine development.

Antibodies that recognize class I and class II human leukocyte antigens (HLA) currently represent a contraindication at multiple stages of the organ transplant process. Prior to transplantation, patients who have been sensitized to produce HLA-specific antibodies typically wait longer to receive a transplant. Post-transplantation, antibodies that recognize the HLA of the donor organ contribute to hyperacute, acute, and chronic rejection of a transplanted organ. However, it is likely that not all antibodies that recognize HLA promote organ failure. A more thorough understanding of anti-HLA antibodies would therefore indicate those immunoglobulins that are truly a contraindication for transplantation.

It has been difficult to evaluate the phenotypic and functional traits of antibodies to any given HLA molecule because anti-HLA humoral responses tend to be polyclonal and these antibodies cannot be readily isolated for individual characterization. Antibody concentration, isotype, epitope specificity, cross-reactivity, and the ability to fix complement have all been implicated as factors that contribute to the pathogenicity of anti-HLA antibodies (6). More advanced tools such as bead-based semi-quantitative assays have recently provided a more definitive indication for these antibodies' HLA specificity. Nonetheless, the complex nature of human sera and the inability to study antibodies reactive against individual HLA antigens continue to cloud the contribution of antibody isotype, concentration, and specificity to transplant rejection.

The current methods of antibody removal only remove antibodies of broad specificity. The PROSORBA® (Cypress Bioscience, San Diego, Calif.) and follow-on IMMUNOSORBA® (Fresenius Medical Care, Waltham, Mass.) products (and others like them) use Protein A to bind a broad range of antibodies. Plasma is filtered through the IMMUNOSORBA® device to rid the majority of IgG antibodies from the sera. However, IgG3 and IgM and other subtypes are NOT removed. These current devices provide no method of selecting between “wanted” and “not-wanted” antibodies.

Therefore, there exists a need in the art for improved devices that selectively remove anti-MHC/HLA antibodies from a sample, as well as methods of production and use thereof, that overcome the disadvantages and defects of the prior art.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic representation of a soluble HLA class II trimolecular complex produced in accordance with the presently disclosed and claimed inventive concept(s).

FIG. 2 is a schematic diagram of a method of producing the soluble HLA (sHLA) class II trimolecular complex (of FIG. 1) in accordance with the presently disclosed and claimed inventive concept(s).

FIG. 3 is a schematic diagram of sHLA class II trimolecular complex production in a hollow fiber bioreactor unit.

FIG. 4 graphically depicts the production of sHLA class II DRB1*0103 produced in transfected cells, demonstrating the ability to scale up production from a T175 flask to a hollow fiber bioreactor unit (CELL PHARM®).

FIG. 5 graphically demonstrates the ability of commercially available monoclonal antibodies (mAb) and patient sera to specifically detect the sHLA DRB1*0103 produced in FIG. 4.

FIG. 6 graphically depicts the ability to produce multiple different sHLA class II complexes from transfected cells in accordance with the presently disclosed and claimed inventive methods.

FIG. 7 graphically depicts production in a bioreactor of milligram quantities of sHLA class II over time.

FIG. 8 demonstrates quantification of sHLA class II DRB*0103/DRA*0101 (produced in FIG. 7) using electrospray mass spectroscopy.

FIG. 9 illustrates the molecular weight results and analysis of the proteins from FIG. 8 and using electrospray ionization TOF mass spectrometry.

FIG. 10 graphically depicts coupling of soluble DRB1*1101 ZP HLA Class II molecule to a solid support and use thereof to facilitate removal of HLA Class II specific antibodies in an ELISA format. Panel A: a diagram of the consecutive absorption matrix ELISA performed for specific antibody removal. Panel B: absorbance and retentate values from 3 different HLA Class II specific mAb antibodies: L243, OL (One Lambda), and 2H11 were subjected to the consecutive absorbance matrix.

FIG. 11 graphically depicts that DRB1*1101-specific human sera was recognized by soluble DRB1*1101 in an ELISA format.

FIG. 12 graphically depicts that soluble DRB1*1101 can be coupled to SEPHAROSE® and used to absorb HLA Class II specific antibody, 9.3F10. Panel A: soluble DRB1*1101 was coupled to SEPHAROSE® Fast Flow and packed into a gravity column. mAb 9.3F10, which has DR reactivity, was passed over the column and flow thru was collected as fractions. Then the mAb was eluted using DEA (diethanolamine) buffer, pH 11.3, was added to the column, and fractions were collected. Panel B: two separate ELISAs for total mouse IgG and human HLA were also performed on the Flow Thru and Eluate to detect specific antibodies versus HLA proteins that might have been eluted off the column.

FIG. 13 graphically depicts that antibodies contained in human sera specific for DRB1*1101 can be removed by a DRB1*1101 specific column. Donor #1 sera was passed over the DRB1*1101 SEPHAROSE® column, and two 2 ml fractions of flow thru were collected. To elute, DEA buffer pH 11.3, was added to the column, and two 2 ml fractions were collected. Panel A: a direct DRB1*1101 ELISA was performed to detect the amount of DRB1*1101 specific antibodies that were left in the flow thru and eluate. Panel B: a total human IgG sandwich ELISA was also performed to evaluate passage of total human IgG.

FIG. 14 graphically depicts that soluble DRB1*1101 coupled SEPHAROSE® is specific for DRB1*1101 and not other DR alleles. Donor #2 sera was passed over the same DRB1*1101 column in the same manner as FIG. 13, and two fractions of the flow thru and one fraction of the eluate were evaluated for multi-allele DR reactivity.

FIG. 15 depicts the nucleic acid (SEQ ID NO:1) and amino acid (SEQ ID NO:2) sequences of a DRA*0101 alpha chain-leucine zipper construct. The highlighted sequence encodes a linker that connects DRA1*0101 allele's sequence to the leucine zipper motif's sequence. The underlined sequence encodes the leucine zipper motif.

FIG. 16 depicts the nucleic acid (SEQ ID NO:3) and amino acid (SEQ ID NO:4) sequences of a DRB1*0401 beta chain-leucine zipper construct. The highlighted sequence encodes a linker that connects DRB1*0401 allele's sequence to the leucine zipper motif's sequence. The underlined sequence encodes the leucine zipper motif.

FIG. 17 depicts the nucleic acid (SEQ ID NO:5) and amino acid (SEQ ID NO:6) sequences of a DRB1*0103 beta chain-leucine zipper construct. The highlighted sequence encodes a linker that connects DRB1*0103 allele's sequence to the leucine zipper motif's sequence. The underlined sequence encodes the leucine zipper motif.

FIG. 18 illustrates the construction of sHLA-DR11. A) The transmembrane domains of the alpha (DRA1*01:01) and beta (DRB1*11:01) chains were deleted and replaced by a 7 amino acid linker followed by leucine zipper ACIDp1(LZA) and leucine zipper BASEp1 (LZB), respectively. B) Amino acid sequences for the mature DRA1*01:01 and DRB1*11:01 constructs. Red letters represent the sequence covered from the MS analysis. Underlined letters show the amino acid sequence for the leucine zipper domains.

FIG. 19 illustrates removal and recovery of L243 with a sHLA-DR11 column. A) A280 values for the fractions obtained from the flow through and elution of the sHLA-DR11 column. B) Class II reactivity of the eluted L243 antibody. The raw MFI for each individual HLA complex tested is shown, and the results are grouped together by loci.

FIG. 20 illustrates the specific removal of anti-HLA-DR11 antibodies using the sHLA-DR11 column. A, B) Representative class II HLA reactivities in the starting sera obtained from two sensitized donors, (A:Donor1, B:Donor2). HLA types are color coded by locus (DR11:black, other DR:shades of blue, DQ: shades of red, DP:green). Data are shown as background corrected MFI (BCMFI). C, D) Anti-HLA reactivity of fractions in the column flow-through and eluate from Donor 1 (C) and Donor 2 (D) were analyzed as in A and B. Each trace shows the reactivity profile for a different class II HLA type as shown in the figure legend. HLA types are color coded as in A and B.

FIG. 21 illustrates removal of complement and non-complement fixing antibodies. A) Complement dependant cytolysis of HLA-DR11 positive cells (C433, C418, C428, C423) using anti-HLA-DR11 antibodies. Mean percent cell death is calculated as described in the materials and methods. Starting serum is shown in blue, flow through in red, and eluate in green. Error bars represent the standard deviation from three independent experiments. Significant differences in mean values are shown and were determined by a one way ANOVA (analysis of variance) with a Turkey post-hoc test (p<0.05). B) Representative fluorescent microscope images used for the quantitative analysis in A. Dead cells are red (ethidium bromide) and viable cells are green (acridine orange).

FIG. 22 illustrates isotype profiles of purified anti-HLA-DR11 antibodies. Antibody isotypes in the starting sera, flow through, and eluate were quantified using a LUMINEX®-based ELISA and expressed as a percentage of total antibody.

FIG. 23 illustrates removal of anti-HLA-DR11 antibodies from sensitized sera. The starting sera from two sensitized donors were tested for class II reactivity using a single antigen bead assay. Once the sera were passed over the sHLA-DR11 column, the flow through, and eluate from the column were tested using the same class II single antigen bead assay.

FIG. 24 illustrates the coupling efficiencies of two different SEPHAROSE® matrices with class I soluble HLA. 1 mg of sHLA-B was added to 1 ml of either CNBr-activated or NHS-activated SEPHAROSE® 4 Fast Flow matrix. The coupling was allowed to react for 1 hour and was terminated. Coupling efficiency is calculated using the following equation: (coupling efficiency=mg starting sHLA/mg sHLA in solution after coupling).

FIG. 25 illustrates the binding capacities of two different SEPHAROSE® matrices for class I soluble HLA. Saturating quantities of pan class I HLA monoclonal antibody W6/32 was run over 1 ml of coupled matrix (1 mg@1 mg/ml). The matrix was either CNBr-activated or NHS-activated SEPHAROSE® 4 Fast Flow matrix. The sHLA used in this experiment was sHLA-B*07:02. Binding capacity was determined by measuring the quantity of antibody recovered in the elution. To adjust for variations in coupling efficiencies, the data is shown as μg of W6/32 in the elution per mg of sHLA coupled on the matrix.

FIG. 26 illustrates the regeneration capabilities of two different SEPHAROSE® matrices loaded with class I soluble HLA. Saturating quantities of pan class I HLA monoclonal antibody W6/32 was run over 1 ml of coupled matrix (1 mg@1 mg/ml). The matrix was either CNBr-activated or NHS-activated SEPHAROSE® 4 Fast Flow matrix. The columns were then serially loaded and eluted 5 times as indicated on the x axis. Percent of the original (cycle 1) antibody binding capacity is shown for each cycle.

FIG. 27 illustrates the coupling efficiencies of two different SEPHAROSE® matrices with class 11 soluble HLA. 1 mg of sHLA-DR11 was added to 1 ml of either CNBr activated or NHS activated SEPHAROSE® 4 Fast Flow matrix. The coupling was allowed to react for 1 hour and was terminated. Coupling efficiency is calculated using the following equation: (coupling efficiency=mg starting sHLA/mg sHLA in solution after coupling).

FIG. 28 illustrates the binding capacities of two different SEPHAROSE® matrices for class 11 soluble HLA. Saturating quantities of pan HLA-DR monoclonal antibody L243 was run over 1 ml of coupled matrix (1 mg@1 mg/ml). The matrix was either CNBr-activated or NHS-activated SEPHAROSE® 4 Fast Flow matrix. The sHLA used in this experiment was sHLA-DR11. Binding capacity was determined by measuring the quantity of antibody recovered in the elution. To adjust for variations in coupling efficiencies, the data is shown as μg of L243 in the elution per mg of sHLA coupled on the matrix.

FIG. 29 illustrates the regeneration capabilities of two different SEPHAROSE® matrices loaded with class 11 soluble HLA. Saturating quantities of pan HLA-DR monoclonal antibody L243 was run over 1 ml of coupled matrix (1 mg@1 mg/ml). The matrix was either CNBr-activated or NHS-activated SEPHAROSE® 4 Fast Flow matrix. The columns were then serially loaded and eluted 5 times as indicated on the x axis. Percent of the original (cycle 1) antibody binding capacity is shown for each cycle.

FIG. 30 illustrates monoclonal anti-HLA antibody depletion from PBS using a class 1 HLA SHARC (soluble HLA antibody removal column). Saturating quantities of pan class 1 HLA monoclonal antibody W6/32 was run over 65 ml of coupled matrix (24.4 mg at 97 μg/ml). The column was then washed with PBS pH 7.4 and eluted with 0.1 M Glycine pH 11. During the load and wash phase, 11.7 mg passed through the column. During the elution phase, 8 mg of anti body was recovered.

FIG. 31 illustrates polyclonal anti-HLA-A2 antibody depletion from patient plasma with class I HLA-A2 SHARC. 2.5 L of Patient plasma containing anti-HLA antibodies was run over the 65 ml sHLA-A2 SHARC. Plasma pre- and post-SHARC were analyzed using a multiplexed, LUMINEX®-based detection method as described by the manufacturer (LABScreen® Single Antigen, OneLambda, Inc., Canoga Park, Calif.). This individual had multiple HLA specificities, as indicated in the legend. As shown in the figure, anti-HLA-A2 antibodies, as well as serologically related antibodies (B57, B58), were reduced from the starting plasma. Serologically unrelated anti-HLA antibodies (B61, B81, B18, B60) were unchanged from the pre-SHARC plasma as they passed through the SHARC. This demonstrates the specificity of the HLA-A2 SHARC.

FIG. 32 illustrates polyclonal anti-HLA-A2 antibody depletion from patient plasma with HLA-A2 SHARC. 2.5L of Patient plasma containing anti-HLA antibodies was run over the 65m1 sHLA-A2 SHARC. Fractions were collected as the plasma was passed over the SHARC. The resulting fractions were analyzed using a multiplexed, LUMINEe-based detection method as described by the manufacturer. Data is represented by percent reduction in BCMFI (% Reduction in BCMFI=1-(BCMFI of the fraction/BCMFI starting plasma).

FIG. 33 illustrates monoclonal anti-HLA antibody depletion from PBS using a class II HLA SHARC (soluble HLA antibody removal column). Saturating quantities of pan HLA-DR monoclonal antibody L243 was ran over 65 ml of coupled matrix (30.0 mg@120 μg/ml). The column was then washed with PBS pH 7.4 and eluted with 0.1 M Glycine pH 11. During the load and wash phase, 2 mg passed through the column. During the elution phase, 23.1 mg of antibody was recovered.

FIG. 34 illustrates polyclonal anti-HLA-DR11 antibody depletion from patient plasma with HLA-DR11 SHARC. 2.5 L of Patient plasma containing anti-HLA antibodies was run over the 65 ml sHLA-DR11 SHARC. Plasma pre- and post-SHARC were analyzed using a multiplexed, LUMINEX®-based detection method as described by the manufacturer (LABScreen® Single Antigen, OneLambda, Inc., Canoga Park, Calif.). This individual had multiple HLA specificities as indicated in the legend. As shown in the figure, anti-HLA-DR11 antibodies as well as serologically related antibodies (DR13, DR4, DR17) were reduced from the starting plasma. Serologically unrelated anti-HLA antibodies (DQ7, DQ8, DQ9) were unchanged from the pre-SHARC plasma as they passed through the SHARC. This demonstrates the specificity of the HLA-DR11 SHARC.

FIG. 35 illustrates polyclonal anti-HLA-DR11 antibody depletion from patient plasma with HLA-DR11 SHARC. 2.5 L of Patient plasma containing anti-HLA antibodies was run over the 65 ml sHLA-DR11 SHARC. Fractions were collected as the plasma was passed over the SHARC. The resulting fractions were analyzed using a multiplexed, LUMINEX®-based detection method as described by the manufacturer. Data is represented by percent reduction in BCMFI (% Reduction in BCMFI=1-(BCMFI of the fraction/BCMFI starting plasma).

FIG. 36 illustrates the coupling efficiency of soluble class I HLA A*0201 to an NHS-activated SEPHAROSE® Fast Flow Matrix column.

FIG. 37 illustrates a repeatability study evaluating the column profile of FIG. 36 based on absorption units (mAU) to detect proteinaceous material.

FIG. 38 illustrates a repeatability study evaluating the column profile of FIG. 36 based on pH.

FIG. 39 illustrates a repeatability study evaluating the column profile of FIG. 36 based on conductivity to detect changes in buffer phases.

FIGS. 40-42 illustrate a stability evaluation of the column of FIG. 36, wherein the column was exposed to multiple rounds of load-elute-equilibrate cycles with W6/32.

FIG. 43 illustrates a capacity evaluation of the column of FIG. 36, utilizing varying amounts of W6/32.

FIG. 44 illustrates a capacity evaluation of the column of FIG. 36, utilizing varying amounts of Anti-β2m.

FIG. 45 illustrates a capacity evaluation of the column of FIG. 36, utilizing varying amounts of Ant-VLDL (an antibody against an artificial tail introduced into the A*0201 molecule).

FIG. 46 illustrates a binding efficiency evaluation of the column of FIG. 36, using W6/32.

FIG. 47 illustrates a binding efficiency evaluation of the column of FIG. 36, using Anti-β2m.

FIG. 48 illustrates a binding efficiency evaluation of the column of FIG. 36, using Anti-VLDL.

FIG. 49 illustrates a proposed application scenario in accordance with one embodiment of the presently disclosed and claimed inventive concept(s).

### DETAILED DESCRIPTION OF THE INVENTIVE CONCEPT(S)

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, “substantially pure” means an object species is the predominant species present (i.e., on a molar basis it is more abundant than any other individual species in the composition). Generally, a substantially pure composition will comprise more than about 50% percent of all macromolecular species present in the composition, such as more than about 55%, 60%, 65%, 70%, 75%, 80%, 85%, 90%, 95%, and 99%. In one embodiment, the object species is purified to essential homogeneity (contaminant species cannot be detected in the composition by conventional detection methods) wherein the composition consists essentially of a single macromolecular species.

The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “isolated protein” referred to herein means a protein of genomic, cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or, (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “MHC” as used herein will be understood to refer to the Major Histocompability Complex, which is defined as a set of gene loci specifying major histocompatibility antigens. The term “HLA” as used herein will be understood to refer to Human Leukocyte Antigens, which is defined as the major histocompatibility antigens found in humans. As used herein, “HLA” is the human form of “MHC”.

The terms “MHC class I light chain” and “MHC class I heavy chain” as used herein will be understood to refer to portions of the MHC class I molecule. Structurally, class I molecules are heterodimers comprised of two noncovalently bound polypeptide chains, a larger “heavy” chain (α) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3^{rd} extracellular domain of the molecule contains the recognition site for the CD8 protein on the CTL; this interaction serves to stabilize the contact between the T cell and the APC. The invariant light chain (12 kDa), encoded outside the MHC on chromosome 15, includes a single, extracellular polypeptide. The terms “MHC class I light chain”, “β-2-microglobulin”, and “β2m” may be used interchangeably herein. Association of the class I heavy and light chains is required for expression of class I molecules on cell membranes.

Like MHC class I molecules, class II molecules are also heterodimers, but in this case consist of two nearly homologous α and β chains, both of which are encoded in the MHC. The class II MHC molecules are membrane-bound glycoproteins, and both the α and β chains contain external domains, a transmembrane anchor segment, and a cytoplasmic segment. Each chain in a class II molecule contains two external domains: the 33-kDa α chain contains α₁ and α₂ external domains, while the 28-kDa β chain contains β₁ and β₂ external domains. The membrane-proximal α₂ and β₂ domains, like the membrane-proximal 3^{rd} extracellular domain of class I heavy chain molecules, bear sequence homology to the immunoglobulin-fold domain structure. The membrane-distal domain of a class II molecule is composed of the α₁ and β₁ domains, which form an antigen-binding cleft for processed peptide antigen. The peptides presented by class II molecules are derived from extracellular proteins (not cytosolic intracellular peptide antigens as in class I); hence, the MHC class II-dependent pathway of antigen presentation is called the endocytic or exogenous pathway. Loading of class II molecules must still occur inside the cell; extracellular proteins are endocytosed, digested in lysosomes, and bound by the class II MHC molecule prior to the molecule's migration to the plasma membrane. Because the peptide-binding groove of MHC class II molecules is open at both ends while the corresponding groove on class I molecules is closed at each end, the peptides presented by MHC class II molecules are longer, generally between 13 and 24 amino acid residues long. Like class I HLA, the peptides that bind to class II molecules often have internal conserved “motifs', but unlike class I-binding peptides, they lack conserved motifs at the carboxyl-terminal end, since the open ended binding cleft allows a bound peptide to extend from both ends.

The term “trimolecular complex” as used herein will be understood to refer to the MHC heterodimer associated with a peptide. An “MHC class I trimolecular complex” or “HLA class I trimolecular complex” will be understood to include the class I heavy and light chains associated together and having a peptide displayed in an antigen binding groove thereof. The terms “MHC class II trimolecular complex” and “HLA class II trimolecular complex” will be understood to include the class II alpha and beta chains associated together and having a peptide displayed in an antigen binding groove thereof.

The term “MHC moiety” as used herein will be understood to include MHC class I trimolecular complexes, MHC class II trimolecular complexes, and any portion or subunit of MHC class I/class II molecules.

The term “biological sample” as used herein will be understood to include, but not be limited to, serum, tissue, blood, plasma, cerebrospinal fluid, tears, saliva, lymph, dialysis fluid, organ or tissue culture derived fluids, and fluids extracted from physiological tissues. The term “biological sample” as used herein will also be understood to include derivatives and fractions of such fluids, as well as combinations thereof. For example, the term “biological sample” will also be understood to include complex mixtures.

The term “HLA protein” as used herein will be understood to refer to any HLA molecule, complex thereof or fragment thereof that is capable of being expressed on a surface of a non-human cell. Examples of HLA proteins that may be utilized in accordance with the presently disclosed and claimed inventive concept(s) include, but are not limited to, an HLA class I trimolecular complex, an HLA class II trimolecular complex, an HLA class II α chain and an HLA class II β chain. Specific examples of HLA class II α and/or β proteins that may be utilized in accordance with the presently disclosed and claimed inventive concept(s) include, but are not limited to, those encoded at the following gene loci: HLA-DRA; HLA-DRB1; HLA-DRB3,4,5; HLA-DQA; HLA-DQB; HLA-DPA; and HLA-DPB.

The term “mammalian cell” as used herein will be understood to refer to any cell capable of expressing a recombinant HLA protein (as defined herein above). Therefore, any “mammalian cell” utilized in accordance with the presently disclosed and claimed inventive concept(s) must contain the necessary machinery and transport proteins required for expression of MHC/HLA proteins and/or MHC/HLA trimolecular complexes on a surface of such cell. “Mammalian cells” utilized in accordance with the presently disclosed and claimed inventive concept(s) must have (A) machinery for chaperoning and loading MHC/HLA proteins, such as class I and class II proteins; and (B) such machinery must be able to interact and work with human HLA proteins, such as class I and class II proteins. Not all cells express class II MHC protein; only professional immune cells such as but not limited to dendritic cells (DC), macrophages, B cells, and the like express class II proteins. Therefore, when it is desired to express HLA class II protein in a mammalian, non-human cell, such non-human cell must express class II MHC for that species and contain the appropriate machinery for interacting and working with both that species' class II MHC as well as human HLA class II. However, the presently disclosed and claimed inventive concept(s) also includes the use of cells of other lineages that have been induced to express class II MHC, such as but not limited to, cytokines, cells that have been subjected to mutagenesis, and the like.

The term “mammalian cell” as used herein refers to immortalized mammalian cell lines and does not include animals or primary cells. Examples of “mammalian cells” that may be utilized in accordance with the presently disclosed and claimed inventive concept(s) include, but are not limited to, human and mouse DC lines, macrophage lines, and B cell lines.

MHC (major histocompatibility complex) or HLA (Human leukocyte antigen) Class II molecules are found only on a few specialized cell types, including macrophages, dendritic cells and B cells, all of which are professional antigen-presenting cells (APCs). The peptides presented by class II molecules are derived from extracellular proteins (not cytosolic as in class I); hence, the MHC class II-dependent pathway of antigen presentation is called the endocytic or exogenous pathway. Loading of class II molecules must still occur inside the cell; extracellular proteins are endocytosed, digested in lysosomes, and bound by the class II MHC molecule prior to the molecule's migration to the plasma membrane.

Like MHC class I molecules, class II molecules are also heterodimers, but in this case consist of two homologous peptides, an α and β chain, both of which are encoded in the MHC. Class II molecules are composed of two polypeptide chains, both encoded by the D region. These polypeptides (alpha and beta) are about 230 and 240 amino acids long, respectively, and are glycosylated, giving molecular weights of about 33 kDa and 28 kDa. These polypeptides fold into two separate domains; alpha-1 and alpha-2 for the alpha polypeptide, and beta-1 and beta-2 for the beta polypeptide. Between the alpha-1 and beta-1 domains lies a region very similar to that seen on the class I molecule. This region, bounded by a beta-pleated sheet on the bottom and two alpha helices on the sides, is capable of binding (via non-covalent interactions) a small peptide. Because the antigen-binding groove of MHC class II molecules is open at both ends while the corresponding groove on class I molecules is closed at each end, the antigens presented by MHC class II molecules are longer, generally between 15 and 24 amino acid residues long. This small peptide is “presented” to a T-cell and defines the antigen “epitope” that the T-cell recognizes.

Turning now to the presently disclosed and claimed inventive concept(s), anti-MHC antibody removal devices, as well as kits containing same, and methods of production and use thereof, are disclosed and claimed herein. The devices/kits described herein may be utilized for various clinical, diagnostic and therapeutic methods, as described in more detail herein below. The anti-MHC antibody removal device includes a soluble MHC moiety covalently coupled to a solid support. The soluble MHC moiety attached to the solid support is serologically active such that the soluble MHC moiety maintains the physical, functional and antigenic integrity of a native MHC trimolecular complex. When a biological sample is brought into contact with the anti-MHC antibody removal device, anti-MHC antibodies specific for the MHC moiety attach to the soluble MHC moiety and are detected and/or removed from the biological sample.

The soluble MHC moiety may be a class I or class II soluble MHC moiety produced by any methods known in the art or otherwise contemplated herein. In certain embodiments, the soluble MHC moiety is a class I or class II soluble HLA moiety. Non-limiting examples of class I soluble HLA moieties that may be utilized in accordance with the presently disclosed and claimed inventive concept(s) (as well as methods of production and purification thereof) are disclosed in U.S. Ser. No. 09/465,321, filed Dec. 17, 1999; U.S. Ser. No. 10/022,066, filed Dec. 18, 2011 (US Publication No. 2003/0166057, published Sep. 4, 2003); and U.S. Ser. No. 10/337,161, filed Jan. 2, 2011 (US Publication No. 2003/0191286, published Oct. 9, 2033). The entire contents of the above-referenced patent applications are hereby expressly incorporated herein by reference. Non-limiting examples of class II soluble HLA moieties that may be utilized in accordance with the presently disclosed and claimed inventive concept(s) (as well as methods of production and purification thereof) are disclosed in parent application U.S. Ser. No. 12/859,002, filed Aug. 18, 2010, and are disclosed in further detail herein below.

In certain embodiments, the MHC/HLA is purified substantially away from other proteins such that the individual MHC/HLA trimolecular complex maintains the physical, functional and antigenic integrity of a native MHC/HLA trimolecular complex. The functionally active, individual MHC/HLA trimolecular complex may be purified as described herein or by any other method known in the art. Upon attachment to the solid support, the conformation of the functionally active, individual MHC/HLA trimolecular complex is maintained.

Any solid support capable of covalent attachment to the MHC/HLA moiety and capable of otherwise functioning in accordance with the presently disclosed and claimed inventive concept(s) may be utilized. In certain embodiments, the solid support may be selected from the group consisting of a well, a bead (such as but not limited to, flow cytometry bead and/or a magnetic bead), a membrane (such as but not limited to, a nitrocellulose membrane, a PVDF membrane, a nylon membrane, and acetate derivative), a microtiter plate, a matrix (such as a SEPHAROSE® matrix), a pore, plastic, glass, a polymer, a polysaccharide, nylon, nitrocellulose, a paramagnetic compound, and combinations thereof. A non-limiting example of a solid support capable of functioning in accordance with the presently disclosed and claimed inventive concept(s) includes a device (such as a column) that possesses an inlet, an outlet, and a chamber disposed therebetween. The chamber contains an inner surface on which the serologically active soluble MHC moiety is disposed, whereby the inlet is disposed to introduce the biological sample into the chamber. As the biological sample flows through the device, anti-MHC antibodies specific for the serologically active MHC moiety attach thereto and are removed from the biological sample. The flow through collected from the outlet is substantially free of anti-MHC antibodies specific for the serologically active MHC moiety. Particular non-limiting examples of devices of this type include human use devices (HUDs), such as an extracorporeal plasmapheresis HUD.

In certain embodiments, NHS-activated SEPHAROSE® matrix is utilized as the solid support. This matrix immobilizes proteins by covalent attachment of their primary amino groups to the NHS (N-hydroxysuccinimide) activated group to form a very stable amide linkage. This is an important feature for therapeutic uses for the devices and methods described herein, as it prevents leaching of the immobilized MHC/HLA complexes from the substrate/solid support during a therapy (such as but not limited to, the use of the device as an extracorporeal device); leaching of these molecules (as well as fragments and/or subunits thereof) could cause deleterious effects to a patient. In addition to increased stability, the NHS-activated SEPHAROSE® matrix also exhibits increased binding capacity resulting from a 14 atom spacer arm present therein; the spacer arm allows the MHC/HLA to reposition as necessary and thus provide better contact with antibodies.

In certain other embodiments, alternative coupling linkages are utilized. Non-limiting examples of other types of linkages include sugar chemistry, carboxy linkage, sulfur linkage, or any other type of linkage chemistry known in the art or otherwise available to a person having ordinary skill in the art that would allow the coupling of an MHC moiety to a solid support.

In certain embodiments, the presently disclosed and claimed inventive concept(s) uses soluble HLA class I trimolecular complexes produced by the methods described in the U.S. patents/patent applications cited herein above. In a non-limiting example, soluble HLA class I trimolecular complexes that are purified substantially away from other proteins such that the individual soluble class I MHC trimolecular complexes maintain the physical, functional and antigenic integrity of the native class I MHC trimolecular complex are provided. The trimolecular complex comprises a recombinant, individual soluble class I MHC heavy chain molecule, beta-2-microglobulin non-covalently associated with the individual soluble class I MHC heavy chain molecule, and a peptide endogenously loaded in an antigen binding groove of the individual soluble class I MHC heavy chain molecule. These molecules are produced by providing a nucleotide segment encoding a desired individual class I MHC heavy chain that has the coding regions encoding the cytoplasmic and transmembrane domains of the desired individual class I MHC heavy chain allele removed such that the nucleotide segment encodes a truncated, soluble form of the desired individual class I MHC heavy chain molecule. This nucleotide segment may be synthetically produced, or it may be produced by locus-specific PCR amplification of the truncated allele (either from cDNA that has been reverse transcribed from mRNA isolated from a source, or directly from gDNA). The nucleotide segment is then cloned into a mammalian expression vector, thereby forming a construct that encodes the desired individual soluble class I MHC heavy chain molecule. A mammalian cell line is then transfected with the construct to provide a mammalian cell line expressing a construct that encodes a recombinant, individual soluble class I MHC heavy chain molecule, wherein the mammalian cell line is able to naturally process proteins into peptide ligands for loading into antigen binding grooves of MHC molecules, and wherein the mammalian cell line expresses beta-2-microglobulin. The mammalian cell line is then cultured under conditions which allow for expression of the recombinant individual soluble class I MHC heavy chain molecule from the construct, such conditions also allowing for endogenous loading of a peptide ligand into the antigen binding groove of each recombinant, individual soluble class I MHC heavy chain molecule and non-covalent association of native, endogenously produced beta-2-microglobulin to form the individual soluble class I MHC trimolecular complexes prior to secretion of the individual soluble class I MHC trimolecular complexes from the cell. The soluble class I MHC trimolecular complexes are then harvested from the culture while retaining the mammalian cell line in culture for production of additional soluble class I MHC trimolecular complexes, and the individual, soluble class I MHC trimolecular complexes are purified substantially away from other proteins, wherein the individual soluble class I MHC trimolecular complexes maintain the physical, functional and antigenic integrity of the native class I MHC trimolecular complex, and wherein each trimolecular complex so purified comprises identical recombinant, individual soluble class I MHC heavy chain molecules.

In other embodiments, the presently disclosed and claimed inventive concept(s) uses soluble HLA class II trimolecular complexes produced by the methods described herein that provide advancements in the areas of purity, quantity, and applications over existing methods; these methods use recombinant DNA methods to alter the protein in a manner that allows mammalian host cells to secrete the protein. HLA class II is naturally produced as a trimolecular complex that is endogenously loaded with peptide ligands and is bound to the membrane. Obtaining such naturally processed and loaded class II presently primarily proceeds by gathering membrane bound forms. Production of membrane bound class II requires cell populations to be lysed for capture of the complex. This method is known as cell lysate and represents state-of-the-art for natural mammalian HLA production for anti-HLA antibody detection assays. Cell lysate class II products are a mixture of numerous cell surface components, including the membrane anchored HLA class II trimolecular complex and other non-HLA proteins that decorate the cell membrane and that co-purify with HLA. Isolation of the HLA from other cell debris and membrane proteins reduces the yield of HLA class II. When producing HLA class II from detergent lysates, one is faced with either contaminating cell surface proteins and/or low class II protein yield. As an alternative, HLA class II can be obtained from Drosophila Schneider S-2 (insect) cell lines (Novak et al., 1999; and U.S. Pat. No. 7,094,555 issued to Kwok et al. on Aug. 22, 2006) and P. pastoris (yeast) (Kalandadze et al. 1996), whereby soluble forms of the HLA class II molecule have been produced. However, class II produced in insect cells lack the endogenously loaded peptides that are an integral component of the HLA class II native trimolecular complex. The HLA molecules produced in insect cells also lack the native glycosylation of mammalian cells. As insect cells lack mammalian protein glycosylation mechanisms and lack the chaperone complexes needed for natural peptide ligand loading, there is a reluctance to utilize class II proteins from insects for clinical applications.

Thus, certain embodiments of the presently disclosed and claimed inventive concept(s) use HLA class II produced by secretion from mammalian cells as a means to produce a native trimolecular complex free of contaminating membrane proteins. Through HLA class II secretion from mammalian cells, a pure product in which the predominant species is the desired HLA class II trimolecular complex is produced. A pure, secreted molecule simplifies and enables downstream purification. Soluble HLA complexes are conducive to hollow fiber bioreactor production systems, such as but not limited to, the CELL PHARM® system (McMurtrey et al. 2008; Hickman et al., 2003; and Prilliman et al., 1999), as well as other systems designed for recombinant native protein secretion from mammalian cells. Highly concentrated harvests are much “cleaner” than cell lysates, thus allowing for minimal product loss because purification is simplified.

Other embodiments of the presently disclosed and claimed inventive concept(s) may utilize HLA class II trimolecular complexes in native form that have been produced and purified via cell lysate methods; however, the complexes produced by these prior art methods have varying amounts of cell membrane secured to the purified HLA product, thereby creating several challenges for the yield of a homogeneous HLA product as well as problems associated with the use thereof.

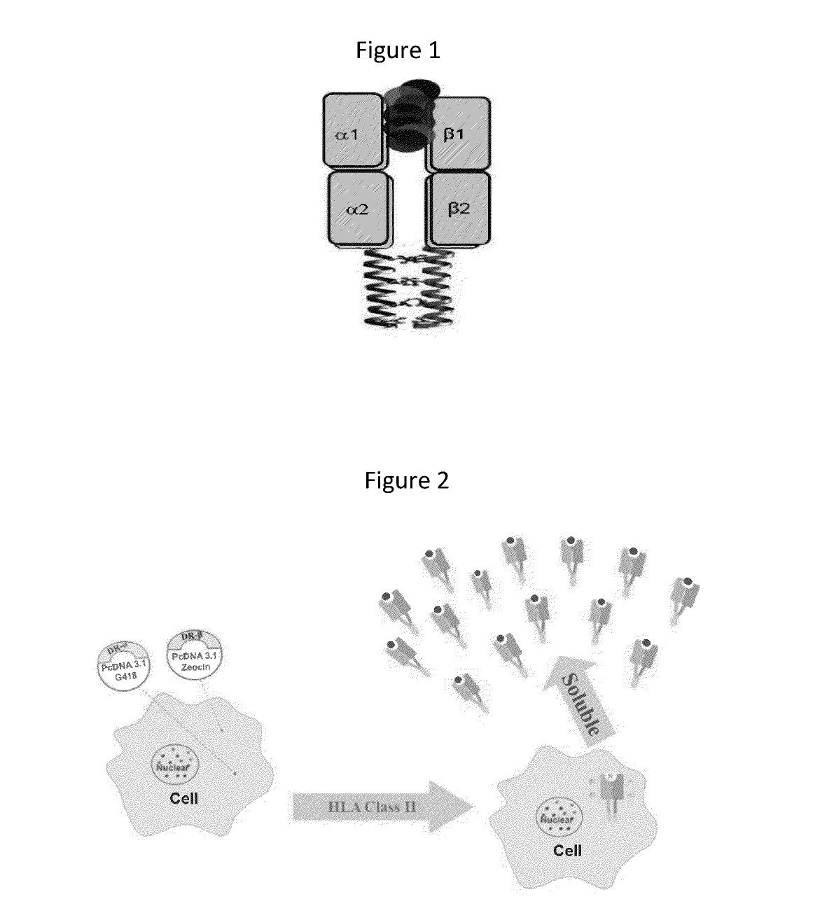

The presently disclosed and claimed inventive concept(s) includes the use of soluble HLA class II trimolecular complexes produced in mammalian cells by a method that solves, in a unique and novel manner, the limitations seen when using cell lysate and insect cell techniques (FIG. 2 illustrates the method of production, while FIG. 1 represents the sHLA trimolecular complexes produced by said method). This production method overcomes the disadvantages and defects of the prior art through the use of a combination of elements; first, each of the α and β chains of the HLA class II complex is truncated such that the domain normally anchoring the complex to the cell surface is removed by recombinant DNA techniques. In native form, the alpha and beta chains of the HLA class II trimolecular complexes rely on the transmembrane domain to maintain a native conformation. While removal of this transmembrane domain facilitates secretion, this removal prevents formation of a trimolecular complex. The sHLA production method removes the transmembrane domain and replaces it with a super secondary structural motif, such as but not limited to, a leucine zipper protein sequence, which serves as a tethering moiety for the class II alpha and beta chains. The super secondary structural motif (such as but not limited to, a leucine zipper) thereby creates adhesion or fusion forces between proteins.

The sHLA production method may further include the recombinant production of the soluble alpha and beta chains of the desired HLA class II in a mammalian cell line. The use of a recombinant mammalian cell line provides two distinct advantages over the prior art: first, production in a mammalian cell line allows the alpha and beta chains of the HLA class II molecule to be glycosylated in the same manner as seen for native HLA class II alpha and beta chains. Second, the mammalian cell line contains the appropriate machinery for natural endocytosis and lysosomal digestion to produce the same peptide ligands as would be produced by a native cell (referred to herein as an “endogenously produced peptide ligand”), as well as the appropriate chaperone machinery for trafficking and loading of the endogenously produced peptide ligands into an antigen binding groove formed between the alpha and beta chains of the HLA class II molecule.

Therefore, the features of (a) glycosylated, soluble HLA class II α and β chains; (b) production in a non-human mammalian cell line (or a human cell line that does not express endogenous class II molecules); and (c) a non-covalently attached, endogenously produced peptide ligand, provide distinct advantages that overcome the disadvantages and defects of the prior art cell lysate and non-mammalian cell production methods.

Endogenously loaded class II is a key element that distinguishes from the prior art. The endogenous peptide allows the class II trimolecular complex to be used in multiple applications not previously possible in soluble forms of the prior art (U.S. Pat. No. 7,094,555, previously incorporated herein by reference; Novak et al., 1999; and Kalandadze et al., 1996). Regarding the currently claimed application method, only a HLA class II in its native trimolecular complex form can properly bind HLA class II specific antibodies. Similarly, the effects of a non-glycosylated HLA molecule on the conformation of class II antibody epitopes when used for HLA specific antibody detection or T-cell solicitation are unknown, but there is some evidence that improper glycosylation disrupts antigen presentation (Guerra et al., 1998). Therefore, the most advantageous format for HLA class II production is to maintain all components in a native form. It has been shown that HLA specific antibody recognition is impacted indirectly by the peptides that are part of the class I complexes (Wilson, 1981). The native binding of HLA specific antibodies is a key element of the presently disclosed and claimed inventive concept(s) when the sHLA described and claimed herein is used as the antigen in an HLA antibody sera screening/removal assay.

In certain embodiments, the presently disclosed and claimed inventive concept(s) utilizes sMHC/sHLA produced by the method described herein below. In the method, a first isolated nucleic acid segment is provided, wherein the first isolated nucleic acid segment encodes a soluble form of an alpha chain of at least one HLA class II molecule, and a second isolated nucleic acid segment is provided, wherein the second isolated nucleic acid segment encodes a soluble form of a beta chain of the at least one HLA class II molecule. The isolated nucleic acid segments may be present in a single recombinant vector, or the isolated nucleic acid segments may be present on two separate recombinant vectors. The coding regions encoding the transmembrane domains of the alpha and beta chains have been removed and replaced with a super secondary structural motif that enables the alpha and beta chains (which previously interacted through their transmembrane domains) to interact. In one embodiment, the super secondary structural motif is a leucine zipper protein sequence that acts as a tethering moiety for the alpha and beta chains.

The isolated nucleic acid segments may be provided by any methods known in the art, including commercial production of synthetic segments. In one embodiment, the nucleic acid segments may be provided by a method that includes the steps of PCR amplification of the alpha and beta alleles from genomic DNA or cDNA. Methods of obtaining gDNA or cDNA for PCR amplification of MHC are described in detail in the inventor's earlier applications U.S. Ser. No. 10/022,066, filed Dec. 18, 2001 and published as US 2003/0166057 A1 on Sep. 4, 2003; and U.S. Pat. No. 7,521,202, issued Apr. 21, 2009; the entire contents of which are hereby expressly incorporated herein by reference. Therefore, while the following non-limiting example begins with gDNA and utilizes PCR amplification, it is to be understood that the scope of the presently disclosed and claimed inventive concept(s) is not to be construed as limited to any particular starting material or method of production, but rather includes any method of providing an isolated nucleic acid segment known in the art.

In one particular embodiment, gDNA is obtained from a sample, wherein portions of the gDNA encode a desired individual HLA class II molecule's alpha chain and beta chain. Two PCR products are then produced: a first PCR product encoding a soluble form of the desired HLA class II alpha chain, and a second PCR product encoding a soluble form of the desired HLA class II beta chain. Each of the PCR products is produced by PCR amplification of the gDNA, wherein the amplifications utilize at least one locus-specific primer having a leucine sequence incorporated into a 3′ primer, thereby resulting in PCR products that do not encode the cytoplasmic and transmembrane domains of the desired HLA class II alpha or beta chains and thus produce PCR products that encode soluble HLA class II alpha or beta chains. The 3′ primer utilized for PCR amplification of the HLA class II alpha chain may incorporate the leucine sequence consistent with the acid sequence of the leucine zipper dimer, while the 3′ primer utilized for PCR amplification of the HLA class II beta chain may incorporate the leucine sequence consistent with the basic sequence of the leucine zipper dimer. However, it is to be understood that the description of the leucine zipper moiety is for purposes of example only, and that the presently disclosed and claimed inventive concept(s) encompasses the use of any super secondary structural motif that enables the alpha and beta chains (which previously interacted through their transmembrane domains) to interact.

Once the isolated nucleic acid segments are provided, they are then inserted into at least one mammalian expression vector to form at least one plasmid containing the PCR products encoding the soluble HLA class II alpha chain and the soluble HLA class II beta chain. It is to be understood that the two nucleic acid segments may be inserted into the same vector or separate vectors.

The plasmid(s) containing the two PCR products are then inserted into at least one suitable immortalized, mammalian host cell line, wherein the cell line contains the necessary machinery and transport proteins required for expression of HLA proteins and/or are able to naturally process proteins into peptide ligands capable of being loaded into antigen binding grooves of HLA class II molecules.

The cell line is then cultured under conditions which allow for expression of the individual soluble HLA class II alpha and beta chains and production of functionally active, individual soluble HLA class II trimolecular complexes, wherein the soluble HLA class II trimolecular complexes comprise a soluble alpha chain, a soluble beta chain and an endogenously loaded peptide displayed in an antigen binding groove formed by the alpha and beta chains. The functionally active, soluble individual HLA class II trimolecular complex maintains the physical, functional and antigenic integrity of a native HLA trimolecular complex.

A primary application of the secreted class II product described herein is the screening of patients awaiting a transplant for anti-HLA antibodies. The requirement for an anti-HLA antibody screening assay is based on the observation that particular events (such as but not limited to, blood transfusion, bacterial infection, and pregnancy) cause one individual to produce antibodies directed against the HLA of other people (Bohmig et al., 2000; Emonds et al., 2000; and Howden et al., 2000). Such anti-HLA antibodies must be detected before a patient receives a transplant, or the transplanted organ will be immediately rejected. Thus, screening for anti-HLA class II antibodies is a prerequisite for organ transplantation.

All transplant patients (approximately 20,000 a year in the U.S.) and all those waiting for a transplant (more than 60,000 a year in the U.S.) must regularly (monthly is preferred) be screened for antibodies that target the HLA of other people. Therefore, these secreted or soluble HLA (sHLA) class II products provide native proteins for quickly and accurately identifying anti-HLA antibodies in those awaiting a transplant. This pre-transplant diagnostic test will prevent rapid organ failure.

The presently disclosed and claimed inventive concept(s) is further directed to a method of producing any of the anti-MHC removal devices described herein above or otherwise contemplated herein. In the method, a serologically active, soluble MHC moiety (as described herein above) is covalently coupled to a solid support (as described herein above). The soluble MHC moiety is attached to the solid support in such a manner that the soluble MHC moiety maintains the physical, functional and antigenic integrity of a native MHC trimolecular complex. In addition, the anti-MHC removal device is constructed so that a biological sample may be brought into contact with the device in a manner that allows the biological sample to interact with the soluble MHC moiety thereof, whereby anti-MHC antibodies specific for the MHC moiety attach to the soluble MHC moiety and are detected and/or removed from the biological sample. The method of producing the anti-MHC removal device may include any steps contemplated or otherwise described herein or otherwise known in the art.

The presently disclosed and claimed inventive concept(s) is further directed to a method for removing anti-MHC antibodies from a biological sample. Such antibody removal is useful, for example, when a patient attacks their transplanted organ with anti-HLA antibodies. Anti-HLA antibodies can also be removed prior to transplantation to enable better outcomes. The removal of antibodies specific for a particular HLA lessens the need for immune suppressing drugs. In the method for removing anti-MHC antibodies from a biological sample, an anti-MHC removal device as described herein above is provided. A biological sample is then brought into contact with the anti-MHC removal device, whereby at least a portion of the antibodies present in the biological sample that are specific for the serologically active, soluble MHC moiety (that is disposed on the surface of the anti-MHC removal device) are removed from the biological sample. The method may further include the step of recovering the biological sample following contact with the anti-MHC removal device, whereby the antibodies specific for the MHC moiety are substantially reduced in the recovered biological sample. The method may further include repeating of the steps of contacting the biological sample to the anti-MHC removal device and recovering the biological sample following said contact. The use of multiple rounds of treatment provides an adequate reduction in antibody titers. In certain embodiments, the recovered biological sample may be substantially free of anti-MHC antibodies specific for the serologically active, soluble MHC moiety of the anti-MHC removal device.

When the anti-MHC removal device includes a device (such as a column) that possesses an inlet, an outlet, and a chamber disposed therebetween (with an inner surface on which the serologically active soluble MHC moiety is disposed), the biological sample is introduced into the chamber via the inlet. The biological sample is then allowed to flow through the device, and at least a portion of the anti-MHC antibodies specific for the serologically active MHC moiety attach thereto and are removed from the biological sample. The flow through is then collected from the outlet, whereby the presence of anti-MHC antibodies specific for the serologically active MHC moiety is substantially reduced.

When the anti-MHC removal device includes a human use device, the method may further include the step of placing the recovered biological sample back into a patient from which it was originally taken.

In certain additional embodiments, the method may further include the step of eluting the anti-MHC antibodies from the anti-MHC removal device. This step may be performed to allow for regeneration and reuse of the anti-MHC removal device. Alternatively, the eluted anti-MHC antibodies may be recovered and used as clinical agents. For example but not by way of limitation, the eluted, recovered anti-MHC antibodies may be utilized for quality control reagents for diagnostics and/or clinical proficiency testing. Thus, compositions that include the eluted, recovered anti-MHC antibodies are also encompassed by the scope of the presently disclosed and claimed inventive concept(s).

The presently disclosed and claimed inventive concept(s) further includes kits useful for removing anti-MHC antibodies from a biological sample. The kit may contain any of the devices described herein, and the kit may further contain other reagent(s) for conducting any of the particular methods described or otherwise contemplated herein. The nature of these additional reagent(s) will depend upon the particular assay format, and identification thereof is well within the skill of one of ordinary skill in the art. In addition, positive and/or negative controls may be included with the kit, and the kit may further include a set of written instructions explaining how to use the kit. The kit may further include a reagent (such as a competitive binding reagent) for elution of the anti-MHC antibodies from the device, thus allowing for regeneration and reuse thereof. Kits of this nature can be used in any of the methods described or otherwise contemplated herein.

Examples are provided hereinbelow. However, the presently disclosed and claimed inventive concept(s) is to be understood to not be limited in its application to the specific experimentation, results and laboratory procedures. Rather, the Examples are simply provided as one of various embodiments and are meant to be exemplary, not exhaustive.

### EXAMPLE 1

### Production of Class II sHLA Trimolecular Complexes for Use in Anti-MHC Removal Devices

This Example is directed to the expression of soluble individual human HLA class II trimolecular complexes in mammalian immortal cell lines. The method includes the use of modifications that alter the endogenous membrane bound complexes in such a way that the membrane bound anchor is disrupted, thereby allowing the cell to secrete the HLA class II trimolecular complexes. In this Example, the Alpha and Beta chain genes encoding HLA class II-DR, HLA-DQ, and HLA-DP were truncated such that the transmembrane and cytoplasmic domains were deleted. At the site of the truncation, a leucine zipper (a tethering moiety) replaced the transmembrane and cytoplasmic that endogenously anchors HLA to the membrane. The leucine zipper allows the HLA to be secreted from the cell while maintaining the class II trimolecular complex native confirmation (FIGS. 1 and 2). The leucine zipper is comprised of an acid segment tailing the class II alpha chain with complementary basic domain tailing the class II beta chain. The acid and basic segments fuse by means of the amino acid leucine being placed every 7 amino acids in the d position of the heptad repeat. The strategy was used by Chang in 1994 to bind the alpha and beta chains of soluble T cell Receptors together in the same fashion.

HLA class II complexes are comprised of two different polypeptide chains, designated α and β. In one method, the alpha and beta constructs were commercially purchased and directly ligated into a mammalian expression vector. In another, the constructs were produced by PCT amplification as described in the paragraph below, followed by purification and ligation into a mammalian expression vector.

Amplification of specific HLA class II genes from genomic DNA or cDNA was accomplished using PCR oligonucleotide primers for alleles at the HLA-DRα HLA-DRA), DRβ (HLA-DRB); DQα (DQA), DQβ (DQB); or DPα (DPA) and DPβ (DPB) gene loci. The beta chain 3′ PCR primer incorporates the leucine sequence consistent with the basic sequence of the leucine zipper dimer. The Alpha chain 3′ primer incorporates the leucine sequence consistent with the acid sequence of the leucine zipper dimer. The truncation of the class II genes through placement of the PCR primers eliminates the cytoplasmic and transmembrane regions, thus resulting in a soluble form of HLA class II trimolecular complex with a leucine zipper moiety.

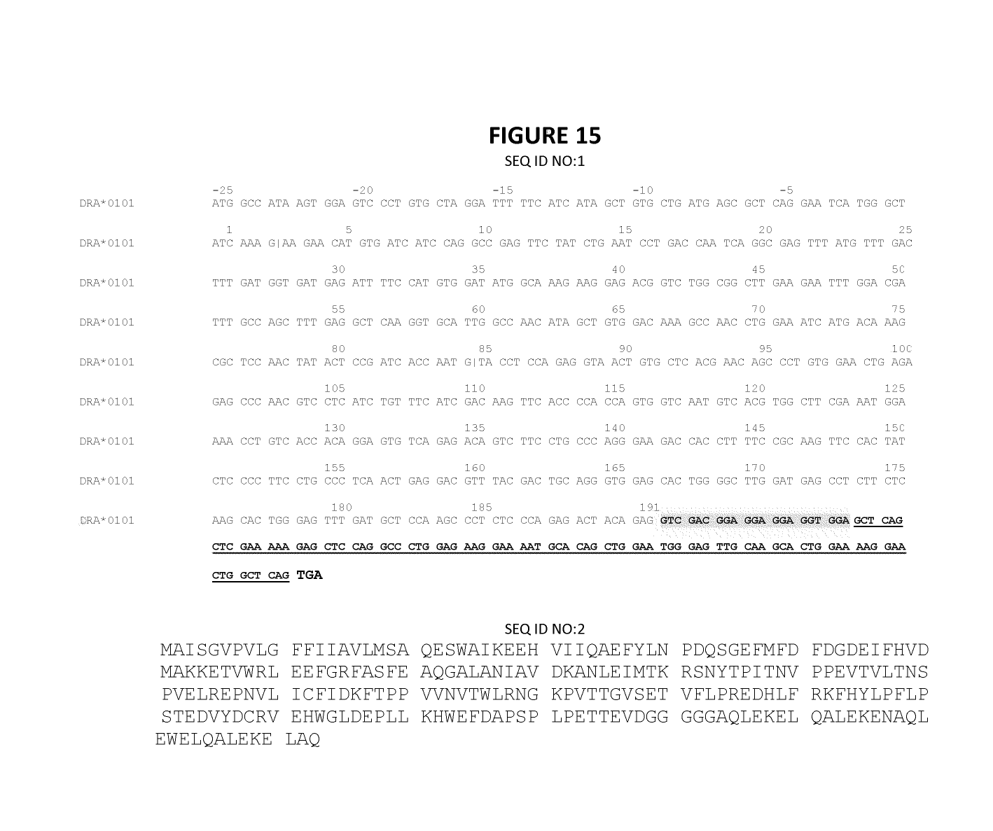

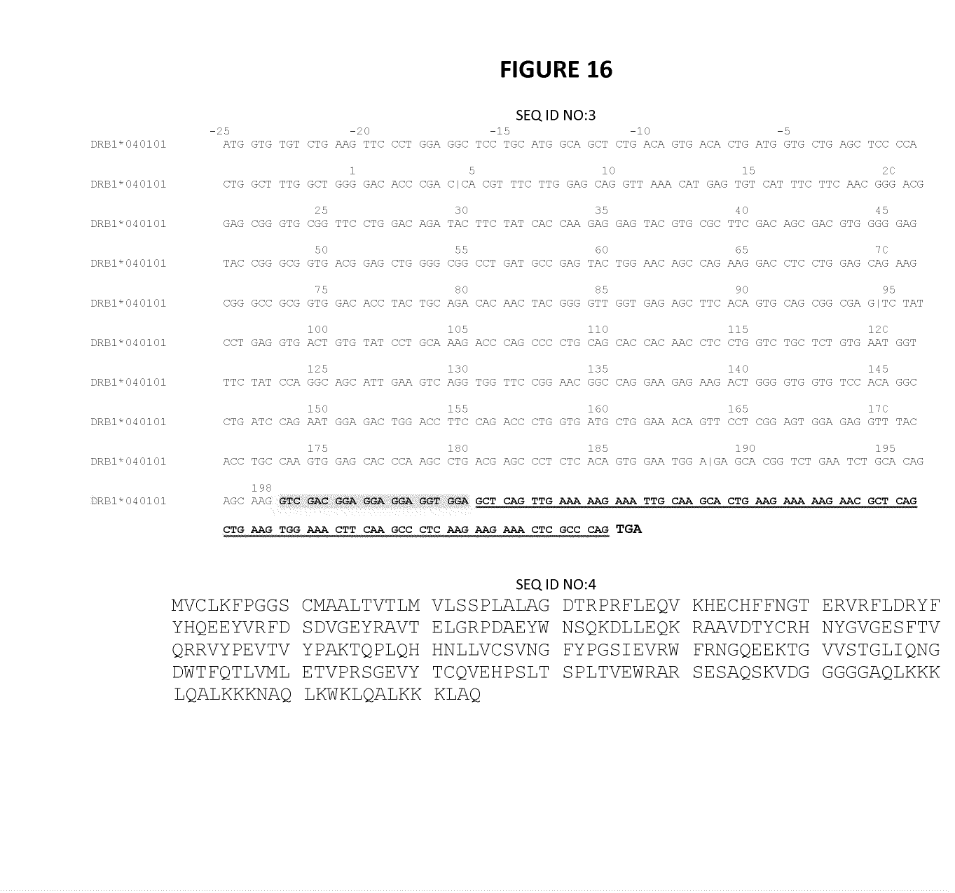

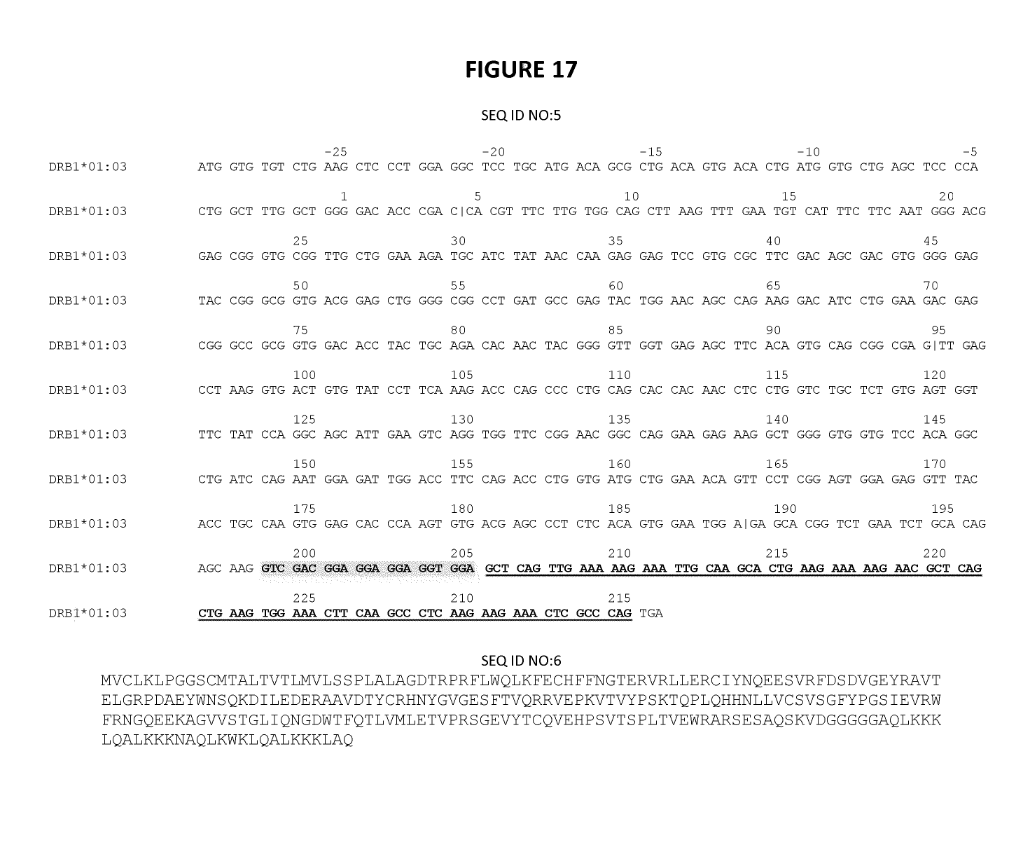

FIGS. 15-17 represent constructs used in the methods of sHLA production of the presently disclosed and claimed inventive concept(s). FIG. 15 illustrates the nucleic acid and amino acid sequences for a DRA1*0101 alpha chain-leucine zipper construct (SEQ ID NOS:1 and 2, respectively). FIG. 16 illustrates the nucleic acid and amino acid sequences for a DRB1*0401 beta chain-leucine zipper construct (SEQ ID NOS:3 and 4, respectively). FIG. 17 illustrates the nucleic acid and amino acid sequences for a DRB1*0103 beta chain-leucine zipper construct (SEQ ID NOS:5 and 6, respectively).

The constructs were then inserted into a mammalian expression vector. In one instance, the alpha chain was cut with one set of restriction enzymes, while the beta chain was cut with another set of restriction enzymes. The purified and cut alpha chain amplification products were ligated into the mammalian expression vector pcDNA3.1. Next, this ligated vector containing the sHLA class II alpha gene was transformed into E. coli strain JM109. The bacteria were grown on a solid medium containing an antibiotic to select for positive clones. Colonies from this plate were picked, grown and checked to contain insert. Plasmid DNA was isolated from the identified positive clones and subsequently DNA sequenced to insure the fidelity of the cloned alpha gene.

The alpha vector was re-cut using a second set of restriction enzymes which facilitate directional cloning of the purified beta PCR product. The final ligation product consisted of both alpha and beta clones. Plasmid DNA was then isolated from positive clones, and the beta genes were DNA sequenced from these clones.

Plasmid DNA for the alpha and beta class II alleles was prepared and DNA sequenced to confirm fidelity of the amplified class II genes. Log phase mammalian cells and the plasmid DNA were mixed in a plastic electrocuvette. This mixture was electroporated, placed on ice and resuspended in media. Special optimization was required for the electroporation step to enable successful enablement of the presently disclosed and claimed inventive concept(s). Standard electroporation procedures were unsuccessful in extensive trials by the inventors and as reported by other labs in publications.

After incubation for 2 days at 37° C. in a CO₂ incubator, the cells were subjected to selection with the antibiotic. First cells were counted and viability was determined. The cells were then resuspended in conditioned complete media. Next, cells were placed into each well of a 24-well plate and left to undergo selection. Supernatant from each well was taken, and an ELISA assay was performed to determine sHLA class II production. High producers were expanded and cryopreserved for large-scale production.

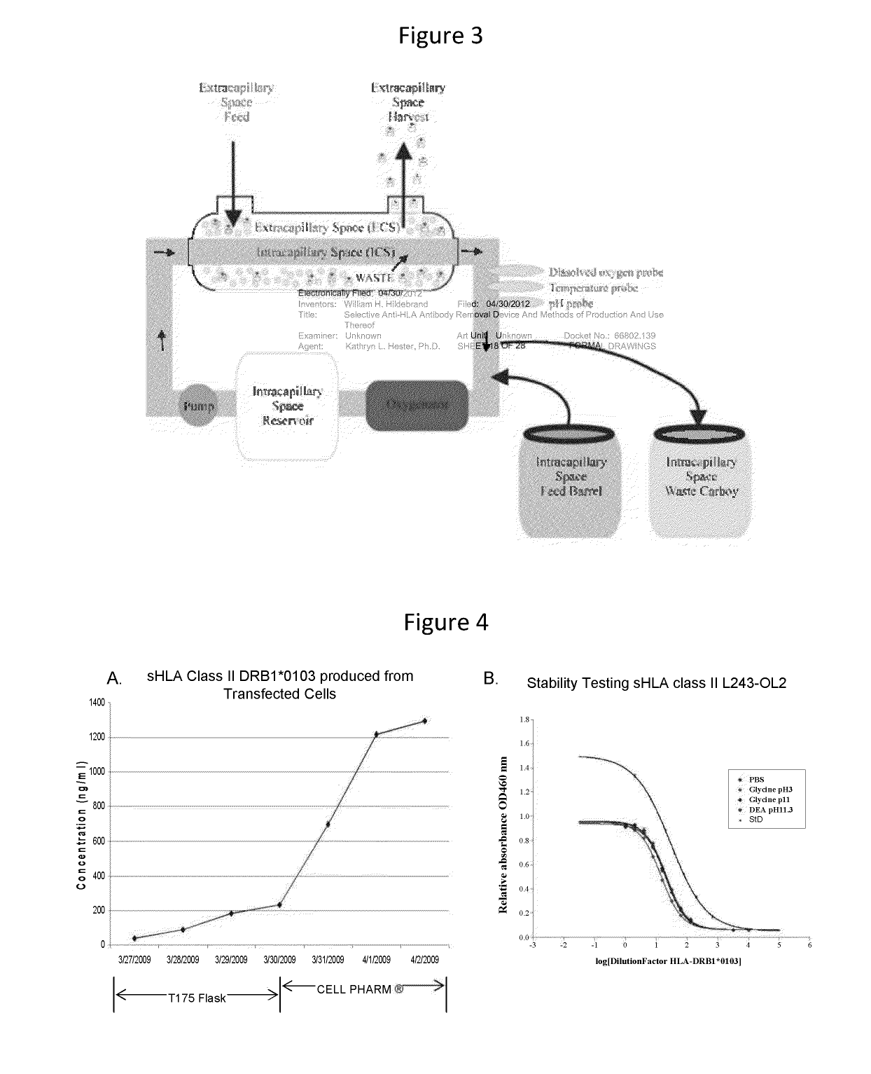

Prior to culture in CELL PHARM® bioreactors, the cellular growth parameters (pH, glucose, and serum supplementation) for each line was optimized for growth in bioreactors. Approximately 8 liters of naïve or pathogen infected sHLA-secreting class II transfectants were cultured in roller bottles in culture media supplemented with penicillin/streptomycin and serum or ITS (insulin-transferrin-selenium) supplement. The total volume of cells cultured was adjusted such that approximately 5×10⁹ cells were obtained. Cells were pelleted by centrifugation and resuspended in 300 ml of conditioned medium in a CELL PHARM® feed bottle. Cells and conditioned medium were inoculated through the ECS feed pump of a Unisyn CP2500 CELL PHARM® into 30 kDa molecular-weight cut-off hollow-fiber bioreactors previously primed with media supplemented with penicillin/streptomycin and serum or ITS. The culture of cells inside the CELL PHARM® was continued with constant monitoring of glucose, pH and infection. Medium feed rates were monitored and adjusted to maintain a glucose level of 70-110 mg/dL. FIG. 3 provides an overview of the CELL PHARM® bioreactor system; the sHLA secreting cells and their sHLA product were contained within the extra capillary space (ECS) of the hollow fiber bioreactor. Nutrients and gases for the cells were provided by recirculated medium.

FIG. 4A illustrates the increased production of sHLA class II DRB1*0103 produced from transfected cells when scaled up to the bioreactor production. The sHLA was purified from the cell supernatant with the specific anti-HLA class II antibody L243 coupled to CNBr-activated SEPHAROSE® 4B, and the protein concentration determined by a micro-BCA protein assay, UV absorbance and ELISA. The sHLA class II titer of a typical production run was found to be approximately 4-5 mg/liter of growth media. FIG. 4B illustrates that these trimolecular complexes were very stable in a wide variety of buffers and at a wide range of pH concentrations using monoclonal antibody L243, which reacts with virtually all DR HLA proteins. L243 is a murine IgG2a anti-HLA-DR monoclonal antibody previously described by Lampson & Levy (1980); said monoclonal antibody has been deposited at the American Type Culture Collection, Rockville, Md., under Accession number ATCC HB55.

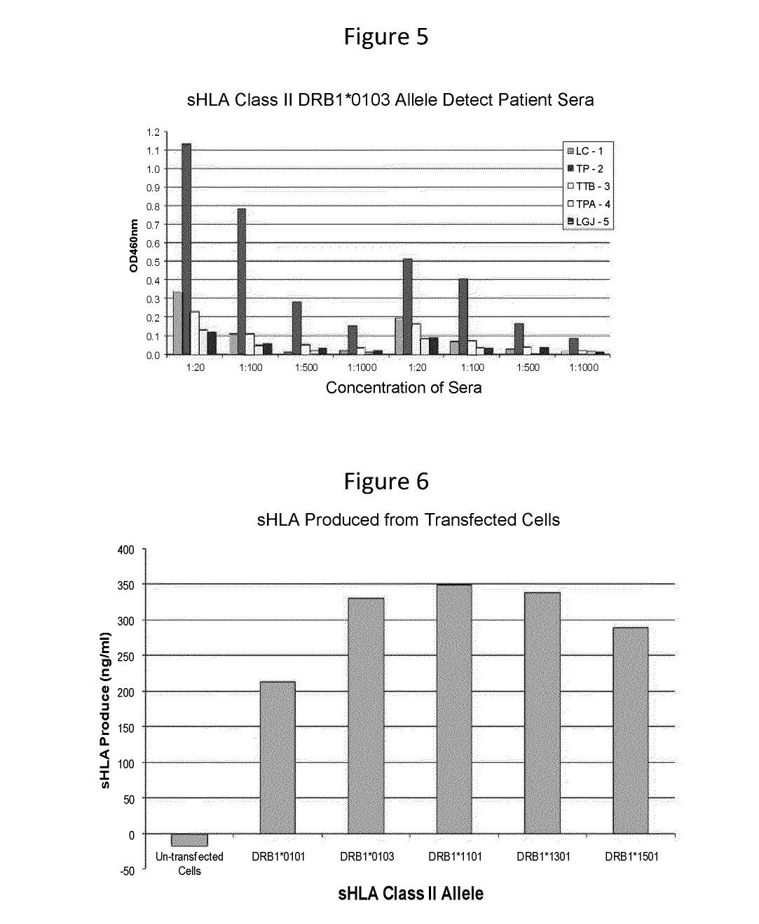

In FIG. 5, the serologic integrity of the purified sHLA class II trimolecular complexes was confirmed by directly coating the complexes on a plate and exposing the coated complexes to defined commercially available mAbs and patient sera. In addition, comparison of the sHLA with full-length molecules showed no differences in antigenicity.

FIG. 6 illustrates the ability to produce multiple different sHLA class II trimolecular complexes by the methods of the presently disclosed and claimed inventive concept(s). While DRB1*0101, DRB1*0103, DRB1*1101, DRB1*1301 and DRB1*1501 are shown for the purposes of example, multiple other sHLA class II trimolecular complexes have also been produced in milligram quantities in accordance with the presently disclosed and claimed inventive concept(s). Trimolecular complexes from each sHLA DR protein have been detected and quantitated using the L243 ELISA-based assay.

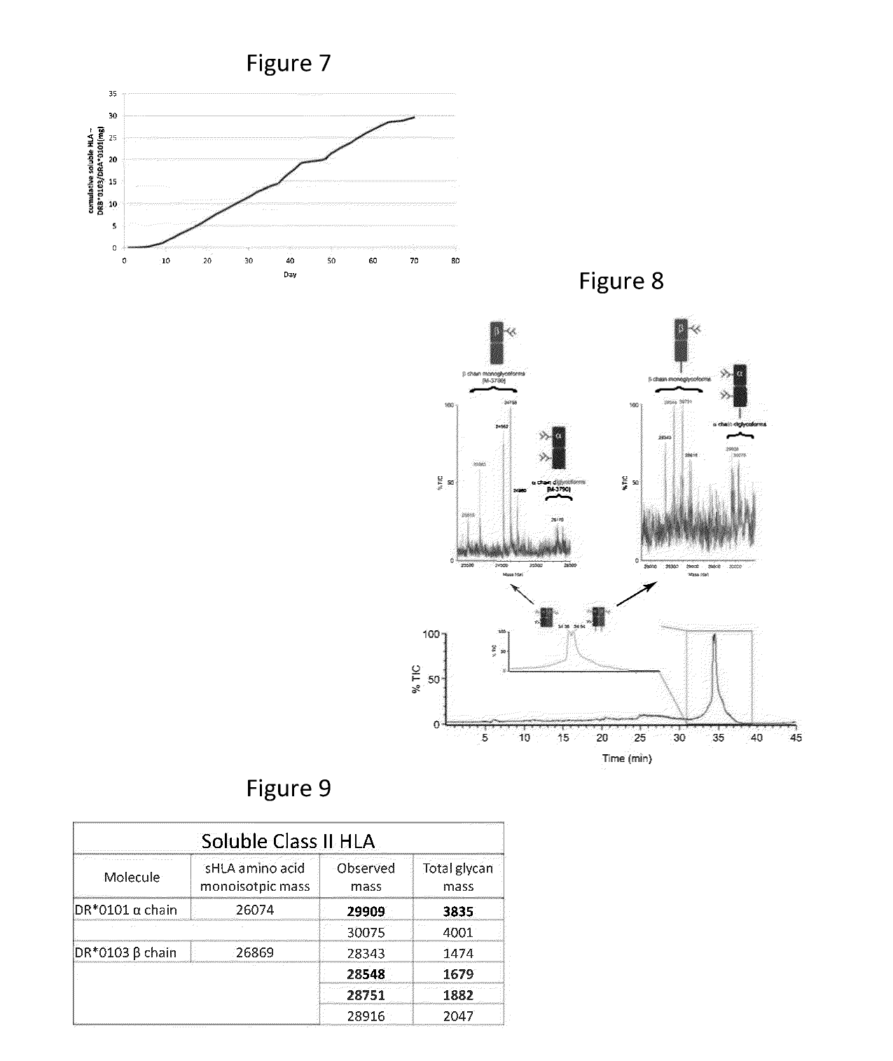

FIGS. 7-9 illustrate another example of sHLA class II production in accordance with the presently disclosed and claimed inventive concept(s). In this example, immortalized cells tranfected with a soluble HLA-DRB*0103/DRA*0101 construct (DRB1*0101 soluble alpha chain with leucine zipper and DRB1*0103 soluble beta chain with leucine zipper) were grown in a roller bottle format until a total 1¹⁰ cells were obtained. The cells were then seeded into the ECS portion of 2 hollow fiber bioreactor units. Cells were grown in DMEM+10% FBS in the ECS and no FBS in the ICS. ECS harvest was collected every day until cells were dead and no longer producing soluble HLA. Protein was quantified using a capture ELISA. For this ELISA an antibody specific for the leucine zipper (2H11) was used as the capture antibody, and an antibody specific for class II HLA (L243) as the detector antibody. Approximately 8mg of soluble HLA was loaded on an affinity antibody (L243) column and eluted in an alkaline buffer (0.1M Glycine, pH 11). Fractions containing soluble HLA were pooled and lyophilized. The lyophilate was resuspended in water/20% acetonitrile and loaded onto a C18 RP-HPLC column. The soluble HLA was then eluted using a 20% to 80% acetonitrile gradient and detected using electrospray ionization TOF mass spectrometry.

As can be seen in FIG. 7, milligram quantities of a soluble form of a single class II HLA heterodimer were produced in the bioreactor format. Additionally, the intact heterodimer was purified with no other contaminating proteins, as determined by LCMS (FIG. 9). This soluble class II contains a monoglycosylated beta chain and diglycosylated alpha consistent with native class II HLA (FIG. 8). Furthermore, the various glycoforms were consistent with the natural variation in sugars that occurs as a protein transits to the cell surface. For a subpopulation of the class II molecules, intracellular proteolytic events removed all but two amino acids of the leucine zipper domain from both the alpha and the beta chains. However, like the full length construct, class II without the leucine zipper domain remain as a heterodimer as both the alpha and beta chains co-elute. These soluble class I and class II HLA proteins are amenable to analysis by mass spectrometry, whereby the purity and identity of these proteins can be confirmed by TOF analysis of molecular weights (FIG. 9).

### EXAMPLE 2

### Use of Class II sHLA for Antibody Removal

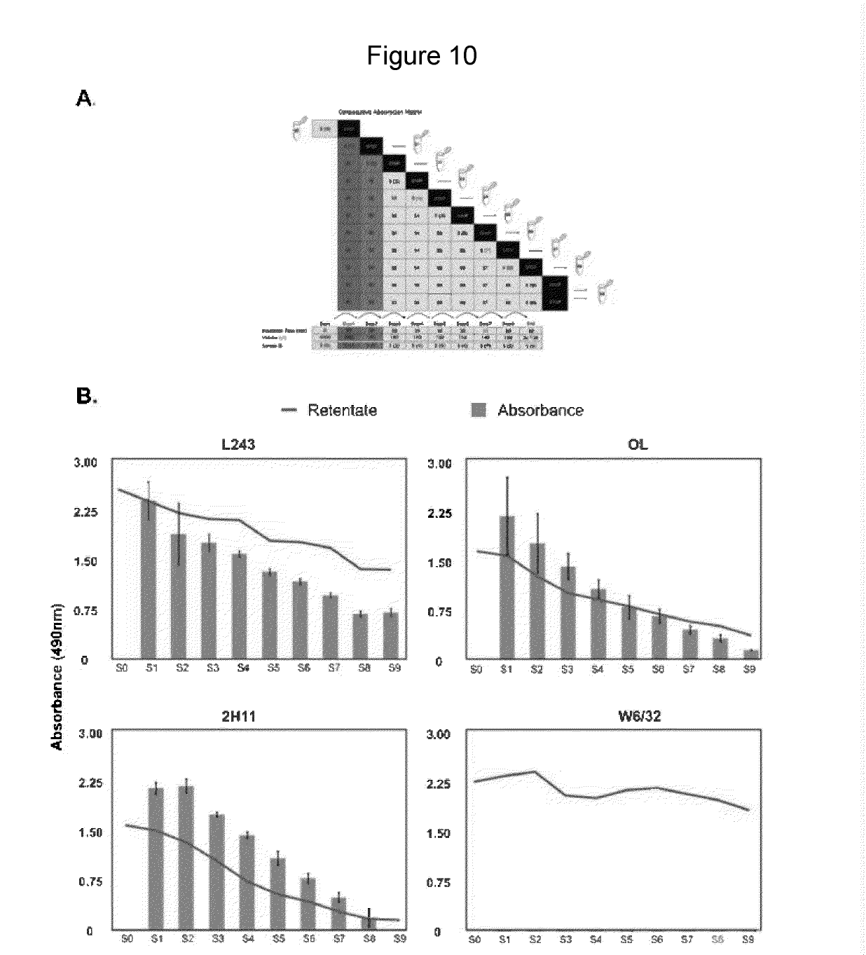

The soluble HLA class II trimolecular complexes of the presently disclosed and claimed inventive concept(s) have also been demonstrated herein to be successfully used in antibody removal techniques, as illustrated in FIGS. 10-14.

FIG. 10 graphically depicts coupling of soluble DRB1*1101 ZP HLA Class II trimolecular complex to a solid support and use thereof to facilitate removal of HLA Class II specific antibodies in an ELISA format. Panel A contains a diagram of the consecutive absorption matrix ELISA performed for specific antibody removal. Briefly, soluble HLA Class II DRB1*1101/DRA1*0101 ZP (labeled as DRB1*1101) was coated to a standard ELISA plate and blocked with BSA. Biotinylated labeled HLAII specific antibodies were then prepared and diluted according to a pre-determined titration for optimal binding, and added to 10 wells as S1. A small portion of this original dilution (204.1) was saved as S(0). The antibody was allowed to bind for 30 minutes at room temperature, after which the entire contents of each well (<204.1) was moved to a corresponding new well (S2), and BSA buffer was added to the S1 wells. This entire process was repeated for a total of 9 sample rounds (S1-S9). For each round, one well was saved in an eppendorf tube for evaluation of the amount of antibody remaining in the retentate solution. These were marked as S(n). After the absorption process was completed, the plate was developed using HRP/OPD peroxidase substrate and plotted as “absorbance.” The retentate samples were also read on a separate ELISA plate in the same manner. These were plotted as “retentate.” Panel B depicts absorbance and retentate values from 3 different HLA Class II specific mAb antibodies: L243, OL (One Lambda), and 2H11 were subjected to the consecutive absorbance matrix. The L243 and OL mAbs, specific for the HLA Class II molecules, and the 2H11 mAb, specific for the zipper tail piece recombinantly added to the soluble HLA Class II molecules, showed a reduction of HLA class II antibodies in the absorption and retentate through each round of the ELISA. One control mAb antibody was included, W6/32, which is specific for HLA Class I molecules, which was not absorbed to the plate and only found in the retentate.

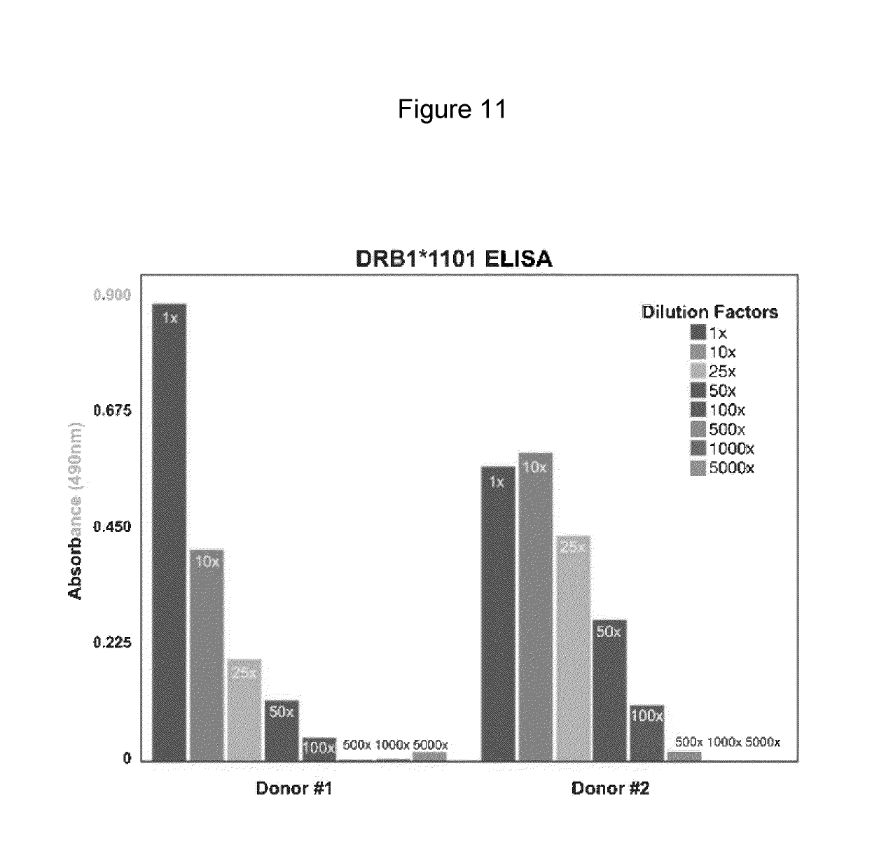

FIG. 11 graphically depicts that DRB1*1101-specific human sera was recognized by soluble DRB1*1101 in an ELISA format. Using soluble HLA Class II DRB1*1101/DRA1*0101 ZP (labeled as DRB1*1101), ELISA plates were directly coated with the HLA Class II soluble allele. Serum samples from two human donors known previously to have DRB1*1101 reactivity were added to the plates in a dilution range from 1× (no dilution) to 5000x. Plates were washed, and a secondary biotinylated goat anti-human IgG antibody was added. Plates were developed using HRP/OPD peroxidase substrate and read at absorbance of 490 nm. Dilution curves for the sera antibody reactivity can be seen for both donors, corresponding to specific avidity for DRB1*1101.

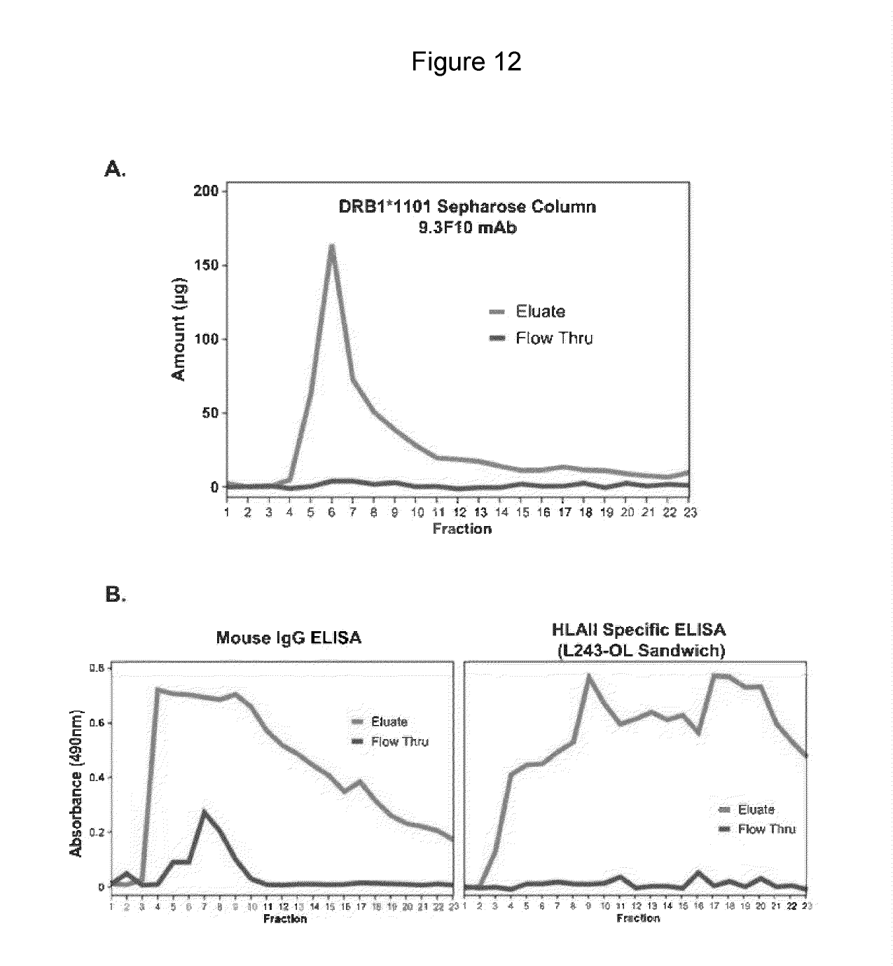

FIG. 12 graphically depicts that soluble DRB1*1101 can be coupled to SEPHAROSE® and used to absorb the HLA Class II specific antibody, 9.3F10. In Panel A, 4 mg of soluble DRB1*1101 was coupled to 1 ml of SEPHAROSE® Fast Flow and packed into a gravity column. A known mixture of 100 μg/ml of mAb 9.3F10 (in 1×PBS), which has DR reactivity, was passed over the column and washed with 1×PBS. A total of 23 200 μl fractions of flow thru were collected, weighed, and measured for OD 280 nm. Values were converted to total amount of protein. To elute the column, roughly 4 ml of DEA (diethanolamine) buffer, pH 11.3, was added to the column, and fractions were collected in 200 μl quantities. The eluate was also weighed, measured at an optical density of 280 nm, and converted to total amount of protein.

In Panel B of FIG. 12, two separate ELISAs for total mouse IgG and human HLA were also performed on the Flow Thru and Eluate to detect specific antibodies (versus HLA proteins) that might have been eluted off the column. Due to the increase in ELISA sensitivity, the minuscule amount of protein seen in the flow thru gave a small peak in the antibody ELISA. Importantly, however, no HLA was seen in the flow thru, but HLA did elute off the column when DEA was added.

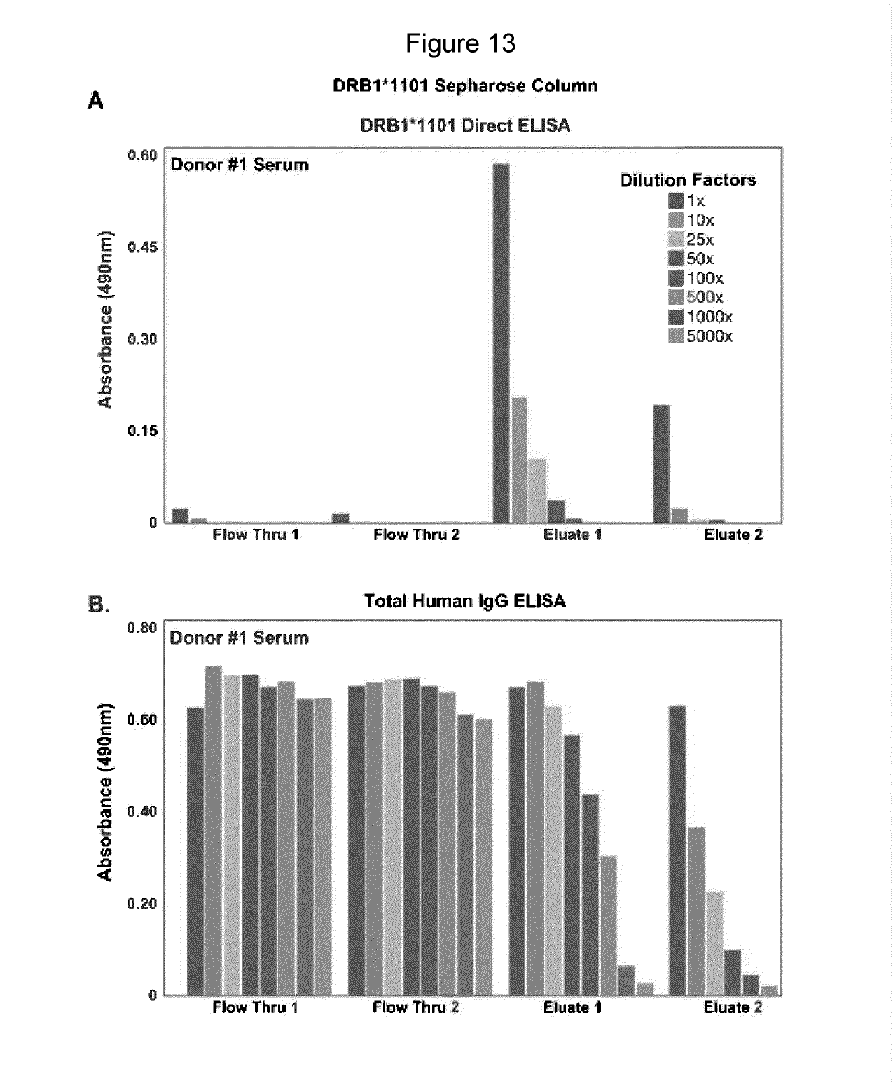

FIG. 13 graphically depicts that antibodies contained in human sera specific for DRB1*1101 can be removed by a DRB1*1101 specific column. Donor #1 sera was passed over the DRB1*1101 SEPHAROSE® column, and two 2 ml fractions of flow thru were collected. To elute, DEA buffer, pH 11.3 was added to the column, and two 2 ml fractions were collected. In Panel A, a direct DRB1*1101 ELISA was performed to detect the amount of DRB1*1101 specific antibodies that were left in the flow thru and eluate. Flow thru and eluate fractions were diluted 1× (no dilution) to 5000× and developed with a biotinylated goat anti-human secondary antibody, followed by HRP/OPD peroxidase substrate. Plates were read at an optical density of 490 nm. In Panel B, a total human IgG sandwich ELISA was also performed to evaluate passage of total human IgG. Total human IgG was seen to pass thru; however only DRB1*1101 antibodies were retained by the column, and only seen once the column was eluted.

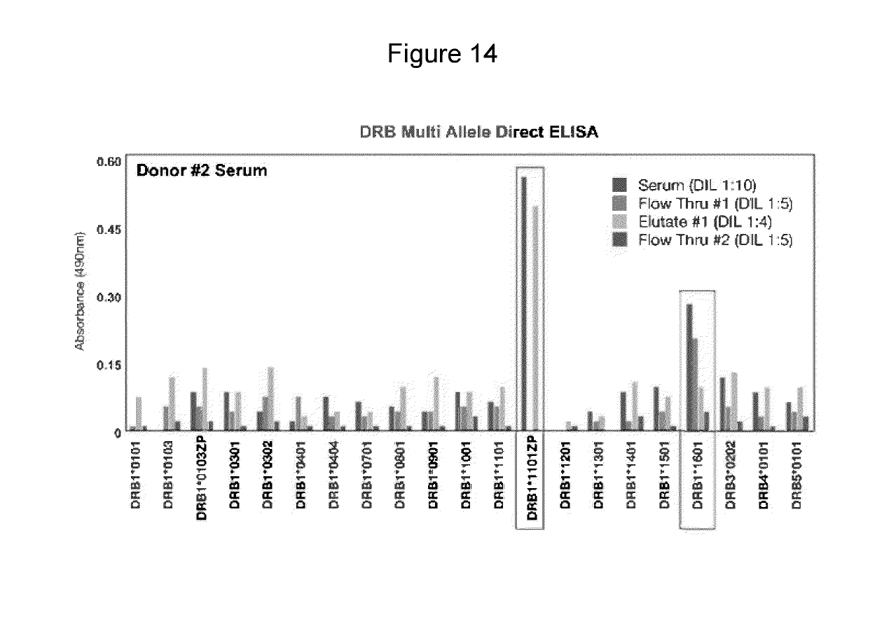

FIG. 14 graphically depicts that soluble DRB1*1101 coupled SEPHAROSE® is specific for DRB1*1101 and not other DR alleles. Donor #2 sera was passed over the same DR1*1101 column in the same manner as FIG. 13, and two fractions of the flow thru and one fraction of the eluate were evaluated for multi-allele DR reactivity. Briefly, multiple alleles of DR from membrane detergent purifications and two DR alleles produced solubly were coated to a 96 well ELISA plate in previously determined optimal amounts for reactivity. Two flow thru fractions and one of the eluate fractions were compared to the original sera sample for reactivity. The second eluate fraction was not evaluated given that most of the specific reactivity was contained in Eluate #1 (FIG. 14). Low reactivity was seen across the board except for the soluble DRB1*1101 (DRB1*1101 ZP) allele, which gave high reactivity to only the sera sample and the eluate but not the flow thrus (first boxed area). The sera also contained strongly reactive antibodies to a second allele, DRB1*1601 (second boxed area), which passed through the flow thru but not the eluate.

Therefore, this Example demonstrates that sHLA class II trimolecular complexes immobilized in a column format can selectively and efficiently remove the vast majority of anti-HLA specific antibodies based on affinity to the bound HLA class II protein in a single pass through, while not removing antibodies that bind to antigenically dissimilar HLA molecules. These data show that a highly specific and efficient antibody removal device can be constructed using the sHLA class II proteins produced in accordance with the presently disclosed and claimed inventive concept(s).

### EXAMPLE 3

### Isolation of HLA-DR11 Antibodies From Sensitized Human Sera

To test the hypothesis that antigen-based isolation of naturally occurring, polyclonal, anti-HLA antibodies would facilitate the characterization of allogeneic anti-HLA antibody responses, appreciable quantities of soluble class II HLA molecules were produced in a native conformation. Next, this unique HLA reagent was used to construct the first reported HLA immunoaffinity column. Donor sera containing a complex mixture of anti-HLA antibodies were then passed over the column. Antibodies specific for a particular class II HLA were retained on the column, and these immunoglobulins were subsequently recovered by elution and characterized. The phenotypic and functional profiling of antigen-specific antibodies represents a substantial advance in the ability to understand how anti-HLA antibodies contribute to organ rejection. A robust application of this technology would distinguish complement-fixing antibodies that represent a contraindication for transplantation from refractory humoral responses that are less of a concern. These immunoaffinity columns constructed with native soluble HLA might also provide a new generation of therapeutic tools for patients with strong antibody reactivity directed towards allogeneic HLA.

### Materials and Methods of Example 3

Patient serum samples: Donor 1 serum was purchased as HLA-DR11 antiserum (Gen-Probe, Inc., San Diego, Calif.). Donor 2 serum was collected from a DR11 sensitized kidney recipient using informed consent according to a protocol approved by the University of Texas Southwestern institutional review board. Donor 2, a 50 year old male, received a kidney graft with a 6/6 mismatch (graft HLA: A2, A3, B62, B51, DR4, DR11). After transplantation donor 2 rejected the graft and developed anti-HLA antibodies. Approximately 5 ml of whole blood was collected and allowed to coagulate. The blood was then centrifuged and the serum was removed from the pellet. Sera were stored at 4° C. until testing.

sHLA-DR11 Protein Production. To produce secreted HLA-DRB11 (sHLA) molecules, α-chain cDNAs of HLA-DRA1*01:01 and HLA-DRB1*11:01 were modified by PCR mutagenesis to delete codons encoding the transmembrane and cytoplasmic domains and add the leucine zipper domains. For DRA*01:01, a 7 amino acid linker (DVGGGGG; SEQ ID NO:7) followed by leucine zipper ACIDp1 was added. For DRB*11:01 the same linker was used, followed by leucine zipper BASEp1 sequence (Busch et al., 2002). sHLA-DRA1*01:01 and sHLA-DRB1*11:01 were cloned into the mammalian expression vector pcDNA3.1(−) geneticin and zeocin respectively (Invitrogen, Life Technologies, Grand Island, N.Y.). The HLA class II deficient B-LCL cell line NS1 (ATCC # TIB-18) was transfected by electroporation simultaneously with sHLA-DRB1*11:01 and DRA1*01:01. Two days post-electroporation cells were transferred into selective growth media containing G418 (0.8 mg/ml) and zeocin (1 mg/ml). Drug resistant stable transfectants were tested for production of sHLA class II molecules by sandwich ELISA using L243 (Leinco Technologies Inc., St. Louis, Mo.) as a capture and class II specific commercial antibody for detection (One Lambda Class II, One Lambda Inc., Canoga Park, Calif.). Individual wells with clonal cell populations were tested for the production of sHLA class II by ELISA and the highest producing clone was expanded in an ACUSYST-MAXIMIZER® hollow fiber bioreactor (Biovest International, Inc., Minneapolis, Minn.). Approximately 25 mg of sHLA-DR11 was harvested from each bioreactor. sHLA-DR11 containing supernatant was loaded on a L243 immunoafffinity column and washed with 40 column volumes of 20 mM phosphate buffer, pH 7.4. sHLA-DR11 molecules were eluted from the affinity column with 50 mM DEA at pH 11.3, neutralized with 1M TRIS pH 7.0, and buffer exchanged and stored at 1 mg/ml in sterile PBS.

Mass spectrometry: 10 μg of purified sHLA-DR11 was reduced and denatured with dithiothreitol (Sigma-Aldrich D0632) and incubated at 95° C. for 5 minutes. The sample was then alkylated with iodoacetamide (Thermo Scientific 89671F), for 1 hour at room temperature. Denatured protein was digested with trypsin using a standard two step digestion protocol (Thermo Scientific 90055). Tryptic peptides were reconstituted in 30% acetic acid/70% ultra-pure water, and loaded onto the ULTIMATE® 3000 HPLC system (Dionex, Thermo Fisher Scientific, Inc., Sunnyvale, Calif.) with a PEPMAP™100 C18 75 μm×15 cm, 3 μm 100 Å reverse phase column. Peptides were eluted and analyzed on a QTOF QSTAR® Elite mass spectrometer (ABI, Thermo Fisher Scientific, MDS Sciex) with Mascot software.

Antibody Removal with DRB1*11:01-Coupled SEPHAROSE® Affinity Columns. For a 1 ml sHLA-DR11 affinity column, SEPHAROSE® 4 Fast Flow (GE Healthcare) was swollen and washed 4 times with ice-cold 1 mM HCl pH 3.0. The swollen matrix was mixed with sHLA-DR11 (4 mg) in bicarbonate coupling solution at a final reaction concentration of 1.6 mg/ml. After the reaction, the matrix was washed three times in coupling buffer and blocked with 0.1M TRIS, pH 8.0. The coupled matrix was then packed into a small 2 ml column.

Either 1 ml of a 200 μg/ml L243 antibody solution or 1 ml of total human sera was applied to the matrix and allowed to be absorbed by gravity. After sample application, 4 ml of PBS pH 7.4 was added. During this loading step, 25 fractions were collected manually, each containing ˜200 μl. Finally, the column was eluted by applying 5 ml of 50 mM DEA pH 11.3. 20 fractions were collected in the elution process and immediately neutralized with 35 μl of 1 M TRIS. For L243, collected fractions were measured by OD₂₈₀ for antibody content. After each procedure, column was mock eluted with DEA, pH11.3 followed by 50 ml of wash buffer (PBS pH 7.4).

Class II HLA Single Antigen Bead Assay and Ig Isotyping. Specificities of anti-HLA antibodies in the pre-column serum, flow through, and eluate were determined using a LUMINEX®-based class II HLA single antigen assay (Gen-Probe GTI Diagnostics), according to manufacturer protocols. Briefly, 40 μl of the bead suspension was incubated with 10 μl of the test sample at room temperature for 30 minutes. Beads were washed and incubated with the detecting antibody at room temperature for 30 minutes, then washed and analyzed on a LUMINEX® 100 analyzer. Data were analyzed using MATCHIT® (Gen-Probe GTI Diagnostics, San Diego, Calif.) and EXCEL® (Microsoft) software. Data for the starting sera and flow through are shown as background corrected median florescence intensity (BCMFI) values based on company defined background levels, which are lot specific and determined by a standard negative sera. With the eluate, there was substantially less background so the background was defined as the minimum bead MFI. For the flow through, the BCMFI values were normalized to the average DQ BCMFI in the starting sera (Tables 1 and 2). The eluate BCMFI values were normalized to the DRB1*11:01 BCMFI in the starting sera (Tables 1 and 2).

For antibody isotyping and quantification the BIO-PLEX PRO™ immunoglobulin isotyping kit (Bio-Rad Laboratories, Inc., Hercules, Calif.) was used according to manufacturer protocols. Briefly, 10-fold serial dilutions of the sample were made and 50 μl of the sample was incubated with 50 μl of the bead suspension for 30 minutes at room temperature. Beads were washed and incubated with the detecting antibody at room temperature for 30 minutes. Last, beads were washed and analyzed on a LUMINEX® 100 (One Lambda, Inc.). Sample MFI values were translated into Ig concentration using the Ig specific standard curves.

Complement Dependant Cytolysis. Complement dependant cytolysis (CDC) was determined using the Lambda Cell Tray: 30 B cell panel (One Lambda, Inc.) Cell lines analyzed were DR11 positive. Cell line class II HLA haplotypes are as follows. C433: DR4, DR11, DR52, DR53, D07. C418: DR4, DR11, DR52, DR53, D07. C423: DR11, DR13, DR52, DQ6, D07. C428: DR11, DR17, DR52, DQ2, DQ7 (One Lambda, Inc.). Lysis was performed on indicated samples according to manufacturer protocols. Rabbit complement was used as a source of complement. After lysis, FLOROQUENCH™ dye (One Lambda, Inc.) was used to differentiate live cells from lysed cells. Live cells and lysed cells were then analyzed using a Nikon TE200-E florescent microscope. Whole well images were generated for each well using the 4× objective lens for both the green filter (excitation: 490nm by 20, emission: 520nm by 38) and the red filter (excitation: 555nm by 28, emission: 617nm by 73). Total florescence in both channels was determined using MetaMorph v 7.5.5.0 and percent cell death was calculated as red florescence/red florescence+green florescence.

### Results for Example 3

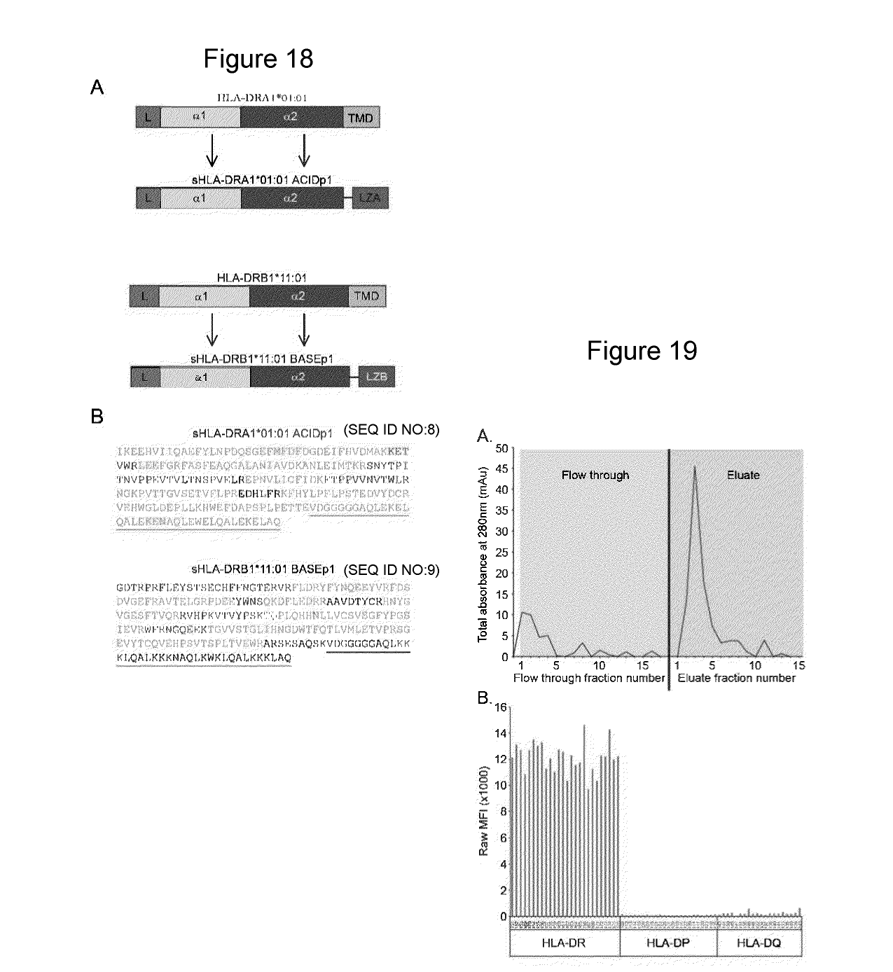

Production and Purification of Soluble Class II HLA. The specific isolation of anti-class II HLA antibodies requires a source of plentiful, native class II HLA. While there are several techniques for obtaining HLA proteins, in this Example, soluble molecules were produced in mammalian cells because these HLA are glycosylated, naturally loaded with ligands, and fully reactive with antibodies. One challenge is that HLA class II exists as an alpha/beta heterodimer and these proteins must be specifically paired to be functional. Previous studies have stabilized the class II soluble HLA heterodimer by replacing the transmembrane and cytoplasmic domains on both the alpha and beta chains with complementary leucine zipper domains (Busch et al., 2002; and Kalandadze et al., 1996), but these studies have only succeeded using non-mammalian cells. Here this approach was used to generate constructs for HLA-DRA1*01:01 and HLA-DRB1*11:01, in which the transmembrane domain is replaced with a 7 amino acid linker followed by an ACIDp1 or BASEp1 leucine zipper domain respectively (FIG. 18A).

A murine cell line was chosen for sHLA-DR11 production, because the inventors hypothesized that the endogenous mouse class II MHC alpha and beta proteins (H2-A^{d}, H2-E^{d}) would not pair with the soluble human class II HLA alpha and beta proteins nor interfere with the intended pairing of the soluble alpha/beta HLA proteins. To confirm that the purified sHLA-DR11 was free from mouse alpha and beta chains, the purified protein was digested with trypsin, and the resulting peptides were subjected to liquid chromatography mass spectrometry (LCMS) analysis. In a BLAST analysis, the peptide sequences showed no matches with the endogenous mouse class II MHC (H2-A^{d}, H2-E^{d}), while peptide sequences were detected from both the alpha and beta chains of the sHLA-DR11 construct transfected into the cells (FIG. 18B). Thus, it was concluded that the desired alpha and beta chain of sDR11 was produced and purified without contamination from other class II MHC subunits.

Column Removal of Anti-HLA Class II Antibodies. In order to test sHLA class II in an immmunoaffinity column format, sHLA-DR11 was purified and coupled to CNBr activated Sepharose-4 Fast Flow solid support matrix. The anti-HLA-DR monoclonal antibody L243 was passed over the affinity column to test whether the sHLA-DR11 complexes remained intact during the coupling process and to measure the binding capacity of the column. Fractions of 200 μl were collected during the loading process (flow through), and bound L243 was eluted intact. Between the flow through and the eluate, 78% (170.6 μg) of the antibody loaded onto the column was recovered, of which 28% (47.8 μg) was in the flow through and 72% (122.9 μg) in the eluate (FIG. 19A). Furthermore the captured and eluted L243 antibody maintained its HLA-DR binding activity and specificity (FIG. 19B). These results demonstrated that HLA-DR11 retained its native conformation when coupled to the affinity column matrix and that a sHLA-DR11 column could be used to remove and recover intact anti-HLA antibodies.

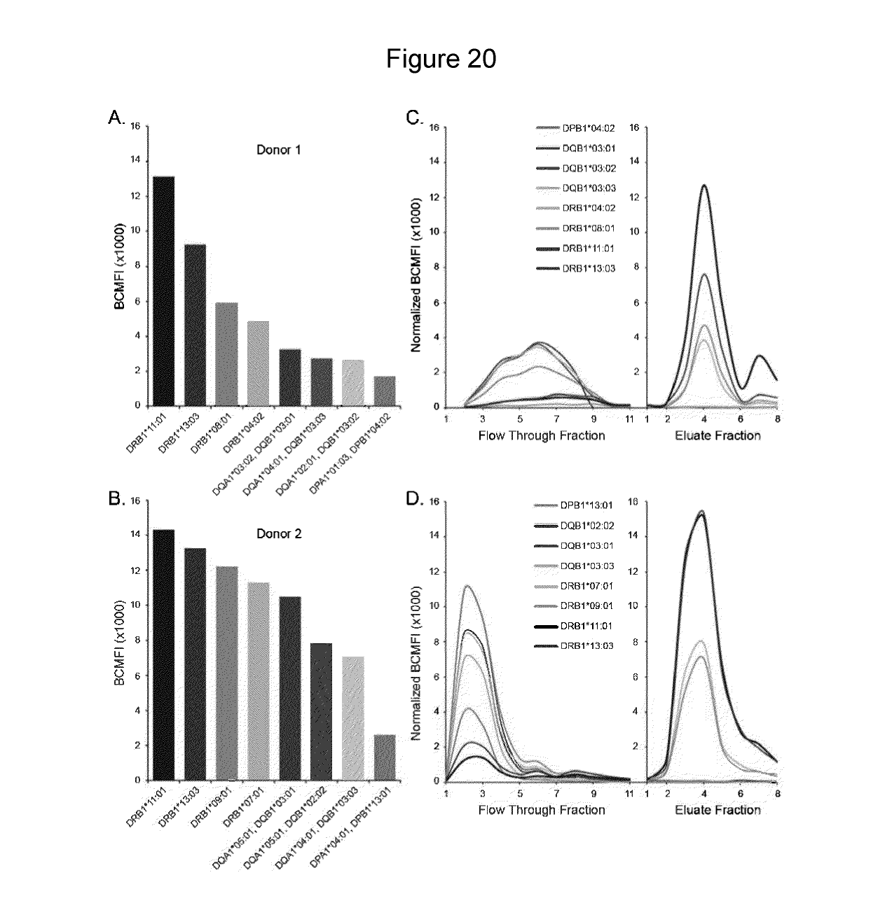

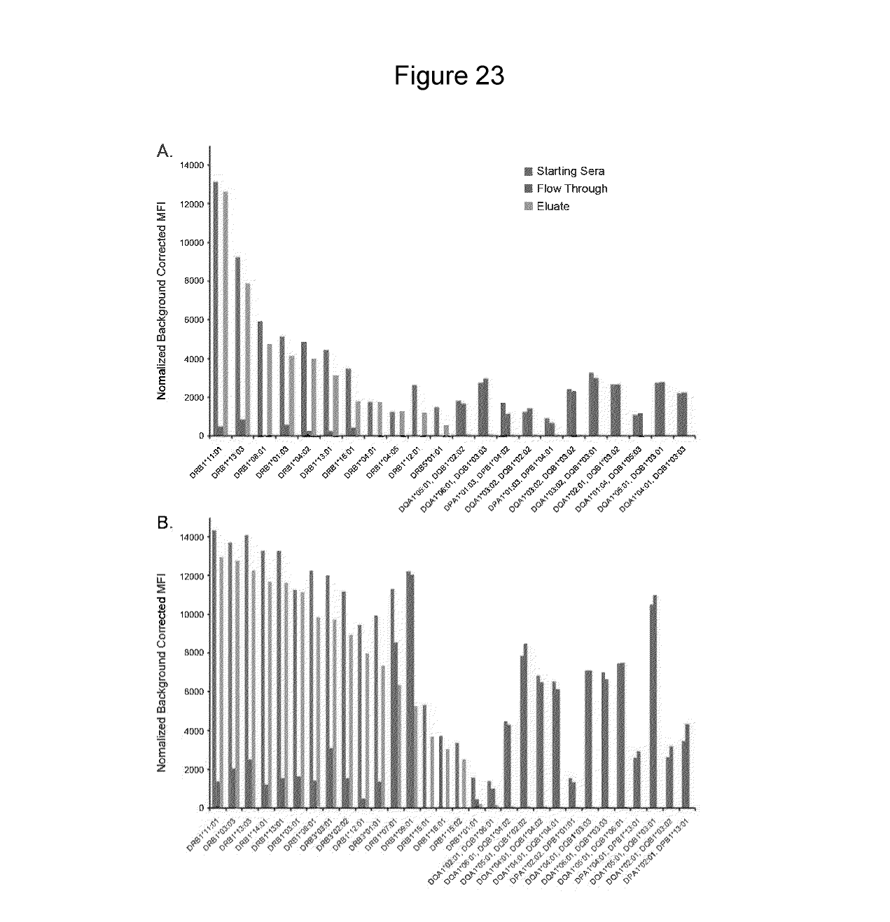

Depletion and Recovery of Anti-HLA-DR11 Antibodies from Patient Sera. Nest, it was tested whether the column could be used to deplete anti-HLA-DR11 antibodies from patient sera. Sera from two DR11 sensitized patients were analyzed for reactivity to multiple class II HLA types in the starting serum (prior to column loading), flow-through, and eluate. Both starting sera contained complex mixtures of polyclonal anti-HLA antibodies reactive with multiple DR and DQ specificities (FIGS. 20A and B). Following passage through the DR11 column, the flow through and eluate from each donor were quite distinct in their patterns of HLA reactivity (FIGS. 20C and D). In the donor 1 serum, HLA-DQ (red) and -DP (green) specific antibodies flowed through the column, while the majority of antibodies to DR11, 13, 8, and 4 were depleted from the serum and subsequently recovered in the eluate. Likewise, in the donor 2 serum, HLA-DQ and -DP specific antibodies passed through the column. However, unlike the donor 1 serum, the majority of DR9 and DR7 specific antibodies from the donor 2 serum flowed through the column, while antibodies to DR11 and DR13 were retained and subsequently eluted. Only small amounts of DR9 and DR7 specific antibodies were recovered in the eluate. All class II HLA reactivity in the starting sera, pooled flow-through (fractions 2-11), and pooled eluate (fractions 2-6) is summarized in FIG. 23.

Prior to column passage, these sera recognized a substantial number of DR specificities (11 HLA-DR in donor 1 and 17 HLA-DR in donor 2). Strikingly, the DR11 column depleted 100% (11/11) of the DR reactive antibodies in donor 1 and 88% (15/17) in donor 2 (FIG. 23, Tables 1 and 2), while no HLA-DQ or DP reactive antibodies were recovered. Thus, the DR11 column removed antibodies to multiple serologically related HLA-DR specificities while antibodies reactive to HLA-DQ and -DP did not bind. These results show that DR11 specific antibodies can be depleted and recovered from patient sera while antibodies reactive with other antigens are not retained.

TABLE 1

| | Pre | Flow Through | | Eluate | |
|---|---|---|---|---|---|
| | Sera | | Normalized* | | Normalized^{†} |
| Bead Antigens | BCMFI | BCMFI | BCMFI | MFI | MFI |
| DRB1*11:01 | 13136 | 517 | 498 | 12063 | 12619 |
| DRB1*13:03 | 9245 | 890 | 857 | 7530 | 7877 |
| DRB1*08:01 | 5945 | 49 | 47 | 4563 | 4773 |
| DRB1*01:03 | 5140 | 612 | 589 | 3964 | 4147 |
| DRB1*04:02 | 4890 | 290 | 279 | 3857 | 4035 |
| DRB1*13:01 | 4447 | 280 | 270 | 2999 | 3137 |
| DRB1*16:01 | 3477 | 456 | 439 | 1705 | 1784 |
| DRB1*04:01 | 1767 | 0 | 0 | 1694 | 1772 |
| DRB1*04:05 | 1243 | 0 | 0 | 1257 | 1315 |
| DRB1*12:01 | 2632 | 0 | 0 | 1172 | 1225 |
| DRB5*01:01 | 1496 | 0 | 0 | 574 | 600 |
| DQA1*05:01, | 1813 | 1728 | 1663 | 97 | 101 |
| DQB1*02:02 | | | | | |
| DQA1*06:01, | 2743 | 3084 | 2969 | 88 | 92 |
| DQB1*03:03 | | | | | |
| DPA1*01:03, | 1729 | 1201 | 1156 | 82 | 85 |
| DPB1*04:02 | | | | | |
| DQA1*03:02, | 1245 | 1472 | 1417 | 75 | 78 |
| DQB1*02:02 | | | | | |
| DPA1*01:03, | 907 | 713 | 686 | 75 | 78 |
| DPB1*04:01 | | | | | |
| DQA1*03:02, | 2422 | 2436 | 2344 | 65 | 68 |
| DQB1*03:02 | | | | | |
| DQA1*03:02, | 3273 | 3109 | 2992 | 51 | 53 |
| DQB1*03:01 | | | | | |
| DQA1*02:01, | 2674 | 2780 | 2676 | 43 | 45 |
| DQB1*03:02 | | | | | |
| DQA1*01:04, | 1113 | 1224 | 1178 | 36 | 38 |
| DQB1*05:03 | | | | | |
| DQA1*05:01, | 2745 | 2891 | 2783 | 25 | 26 |
| DQB1*03:01 | | | | | |
| DQA1*04:01, | 2229 | 2322 | 2235 | 24 | 25 |
| DQB1*03:03 | | | | | |
| Normalization | | | 0.96 | | 1.05 |
| Ratio | | | | | |

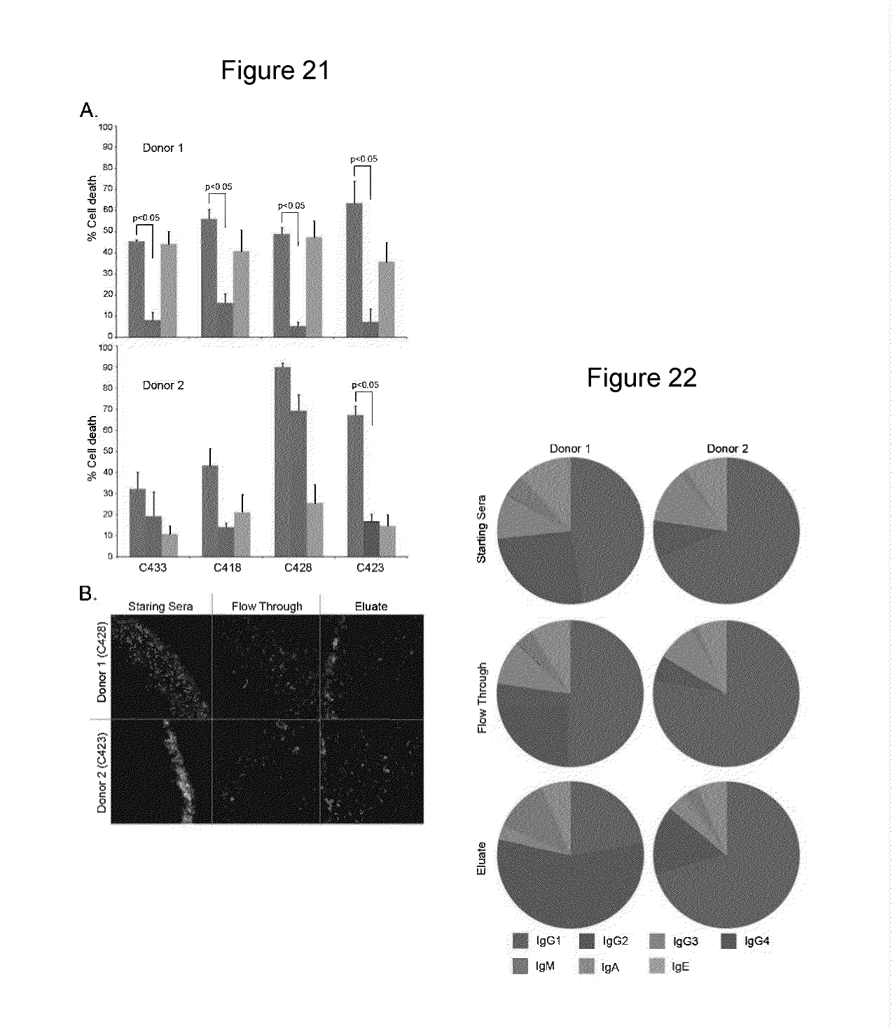

Background corrected MFI values for Donor 1 used to generate FIG. 21A and FIG. 23.
*BCMFI values was normalized to the average DQ BCMFI in the starting sera.
^{†}BCMFI values was normalized to the DRB1*11:01 BCMFI in the starting sera.

TABLE 2

| | Pre | Flow Through | | Eluate | |
|---|---|---|---|---|---|
| | Sera | | Normalized* | | Normalized^{†} |
| Bead Antigens | BCMFI | BCMFI | BCMFI | MFI | MFI |
| DRB1*11:01 | 14320 | 1094 | 1386 | 10721 | 12934 |
| DRB1*03:03 | 13703 | 1618 | 2050 | 10567 | 12748 |
| DRB1*13:03 | 14101 | 1980 | 2509 | 10146 | 12241 |
| DRB1*14:01 | 13267 | 973 | 1232 | 9667 | 11663 |
| DRB1*13:01 | 13268 | 1233 | 1563 | 9622 | 11608 |
| DRB1*03:01 | 11249 | 1285 | 1628 | 9232 | 11138 |
| DRB1*08:01 | 12247 | 1130 | 1431 | 8150 | 9832 |
| DRB3*03:01 | 12014 | 2434 | 3085 | 8065 | 9730 |
| DRB3*02:02 | 11172 | 1216 | 1541 | 7399 | 8926 |
| DRB1*12:01 | 9453 | 390 | 494 | 6599 | 7961 |
| DRB3*01:01 | 9915 | 1073 | 1360 | 6078 | 7333 |
| DRB1*07:01 | 11299 | 6741 | 8543 | 5247 | 6330 |
| DRB1*09:01 | 12218 | 9504 | 12044 | 4370 | 5272 |
| DRB1*15:01 | 5333 | 0 | 0 | 3092 | 3730 |
| DRB1*16:01 | 3729 | 0 | 0 | 2530 | 3052 |
| DRB1*15:02 | 3374 | 0 | 0 | 2076 | 2505 |
| DRB1*01:01 | 1569 | 362 | 459 | 180 | 217 |
| DQA1*02:01, | 1391 | 787 | 997 | 130 | 156 |
| DQB1*06:01 | | | | | |
| DQA1*06:01, | 4460 | 3376 | 4278 | 93 | 112 |
| DQB1*04:02 | | | | | |
| DQA1*05:01, | 7845 | 6681 | 8467 | 90 | 108 |
| DQB1*02:02 | | | | | |
| DQA1*04:01, | 6815 | 5123 | 6492 | 76 | 92 |
| DQB1*04:02 | | | | | |
| DQA1*04:01, | 6534 | 4833 | 6125 | 71 | 86 |
| DQB1*04:01 | | | | | |
| DPA1*02:02, | 1566 | 1049 | 1329 | 69 | 83 |
| DPB1*01:01 | | | | | |
| DQA1*04:01, | 7082 | 5599.5 | 7096 | 67 | 81 |
| DQB1*03:03 | | | | | |
| DQA1*06:01, | 7024 | 5252 | 6656 | 63 | 75 |
| DQB1*03:03 | | | | | |
| DQA1*05:01, | 7463 | 5899.5 | 7476 | 59 | 71 |
| DQB1*06:01 | | | | | |
| DPA1*04:01, | 2607 | 2319 | 2939 | 30 | 36 |
| DPB1*13:01 | | | | | |
| DQA1*05:01, | 10486 | 8673 | 10991 | 28 | 34 |
| DQB1*03:01 | | | | | |
| DQA1*02:01, | 2645 | 2499 | 3167 | 26 | 31 |
| DQB1*03:02 | | | | | |
| DPA1*02:01, | 3463 | 3402 | 4311 | 21 | 25 |
| DPB1*13:01 | | | | | |
| Normalization | | | 1.27 | | 1.21 |
| Ratio | | | | | |

Background corrected MFI values for Donor 2 used to generate FIG. 21B and FIG. 23.
*BCMFI values was normalized to the average DQ BCMFI in the starting sera.
^{†}BCMFI values was normalized to the DRB1*11:01 BCMFI in the starting sera.

Purified HLA-DR11 Antibodies Fix Complement. To evaluate the functional traits of antibodies for HLA-DR11, the complement fixing activity of the donor 1 and donor 2 starting sera, flow through, and eluate were tested. HLA-DR11 positive cells were incubated with starting sera, flow through, or eluate in the presence of complement. Complement dependent cytolysis (CDC) was measured with florescent microscopy. In donor 1 serum, the DR11 column depleted CDC activity to all 4 DR11 target cell types (FIG. 21; C433, C418, C428, C423), and this DR11 specific CDC activity was recovered in the eluate. Thus, anti-DR11 antibodies were necessary and sufficient for CDC activity in patient 1. The donor 2 serum showed heterogeneous reactivity to the different target cell lines in the assay. On some cell lines (C433, C418, C428), the removal of DR11 antibodies did not significantly reduce the CDC activity in the flow through, likely due to complement fixing antibodies directed towards the other HLA present on the target cells. Interestingly, CDC activity on cell line C423 was depleted in both the donor 2 flow through and eluate, indicating that anti-DR11 antibodies were necessary but not sufficient for CDC activity. These data demonstrate that antibodies to individual HLA can be isolated and functionally characterize, and that anti-HLA CDC activity can vary between individuals.

Quantity and Quality of Polyclonal HLA-DR11 Antibodies. The HLA immunoaffinity column provided a unique opportunity to study patient-derived populations of DR11 reactive antibodies. Antibody function is largely dictated by Ig constant region, or antibody isotype. Therefore, the isotype of DR11 reactive antibodies was characterized in patient sera. Several different isotypes were observed in the starting sera, the pooled flow through, and the pooled eluate for both donors (FIG. 22). IgG1 predominated in both the starting sera and in the flow through, with appreciable IgG2, IgA, IgG3, and some IgM present. The isotype profile of antibodies eluted from the DR11 column was diverse in both individuals, with 5 of the 7 Ig isotypes represented in the eluate. In the donor 1 eluate, IgG2 was the most common isotype, with considerable levels of IgG1, IgM, and IgA. In contrast, IgG1 predominated in the donor 2 eluate, with appreciable IgG2 and detectable IgA, IgM, and IgG3. The antibodies in the donor 1 eluate were 56.1% IgG2, 22.3% IgG1 and 11.6% IgM, whereas the donor 2 eluate contained 70.5% IgG1, 15.5% IgG2, and 3.3% IgM (FIG. 22). Both eluate samples showed similar low levels of IgA and IgG3, with IgA comprising 6.6% and 6.3% and IgG3 comprising 3.3% and 4.2% of the eluate for donor 1 and donor 2, respectively. This preliminary dataset suggests substantial heterogeneity can exist in anti-HLA antibody isotype.

The column depleted all detectable anti-HLA-DR activity from the donor 1 serum, allowing the total concentration of anti-HLA-DR antibodies in this patient to be estimated. The pooled eluate of donor 1 contained 17.7 μg/ml of antibody. Assuming the efficiency of antibody recovery from serum was similar to that of mAb L243, and factoring for volume variation, the serum concentration of the anti-HLA-DR antibody in donor 1 was approximately 23.7 μg/ml, or 0.05% of the total Ig. While this may not be representative of concentrations in other donor sera, it demonstrates that these immunoaffinity columns enable, for the first time, the direct quantification of anti-HLA antibodies in patient sera.

### Discussion of Example 3

Donor specific anti-HLA antibodies represent a pre-transplant contraindication and a post-transplant risk for graft loss. While it is clear that antibodies to HLA mediate graft failure and loss, studies also suggest that not all anti-HLA antibodies are detrimental (Wasowska, 2010; and Amico et al., 2009). These observations have sparked great interest in discerning what differentiates pathogenic anti-HLA antibodies from those that are not a threat to transplanted organs. To date, the tools available for studying antibodies to HLA have not been sufficient for characterizing or detecting antibodies that warrant clinical intervention. In this Example, HLA-DR11 immunoaffinity columns were used to characterize patterns of HLA-DR serologic cross-reactivity, to phenotype DR11 reactive antibodies, and to assess the function of antibodies in patient sera. This ability to isolate anti-HLA antibodies is positioned to augment both clinical and basic scientific endeavors by unraveling the complex nature of humoral responses to HLA.

Anti-HLA antibody responses are recognized as polyclonal and heterogeneous. In particular, allogeneic antibody responses to class II HLA are highly cross-reactive, with any given serum reacting to multiple class II HLA (EI-Awar et al., 2007). Indeed, antibodies reactive to the HLA-DR11 column recognized a striking diversity of HLA-DR specificities. The HLA-DR11 column depleted 11 different HLA-DR specificities from the donor 1 serum while 15 HLA-DR specificities were removed from donor 2 (FIG. 23). The HLA-DR11 reactive antibodies purified from donor 1 then reacted with HLA-DR103, 4, 8, 12, 13, 16, and 51 with no reactivity to the remaining 26 DR complexes tested. The pattern of serologic cross reactivity observed for donor 1 was consistent with the recognition of a solvent accessible Asp residue present at position 70 in the beta chain of all recognized HLA-DR complexes but in none of the other HLA-DR complex except HLA-DR7 (EI-Awar et al., 2007). The serologic reactivity pattern for antibodies recovered from donor 2 was more complex; the anti-HLA-DR11 antibodies cross reacted with every HLA-DR tested except for HLA-DR1, 103, 4, 10, 51, and 53. Interestingly, antibodies directed towards HLA-DR7 and HLA-DR9 were split into two groups; those that bound HLA-DR11 and those that did not (FIG. 23). This demonstrates the availability of two (or more) distinct epitopes in the HLA-DR7 and HLA-DR9 reactive antibody pool, only one of which is shared with DR11. These data illustrate the use of HLA immunoaffinity columns to characterize the target epitopes and cross-reactivity of anti-HLA antibodies, and the variability of anti-HLA reactivity profiles from patient to patient.

In addition to deciphering patterns of serologic recognition, HLA-DR11 reactive sera were analyzed for their isotype profile and ability to fix complement. The straightforward relationship between isotype profile and CDC activity in Donor 1 indicated that complement-fixing anti-HLA-DR11 antibodies (i.e., IgG1 and IgM) were responsible for anti-HLA-DR11 CDC activity in the Donor 1 starting serum and that the HLA-DR11 column removed complement fixing activity from the flow through by depleting HLA-DR11-reactive antibodies. The relationship between isotype profile and CDC activity was more complex for Donor 2. The Donor 2 eluate was dominated by non-complement-fixing IgG1, and CDC activity was lost in both the flow-through and eluate. This finding is consistent with antibody synergy, which has been previously described in complement fixation. Murine models of MHC class I mismatch during cardiac transplantation demonstrated that modest amounts of complement-fixing (IgG2a) antibodies to MHC fix complement much more effectively when combined with non-complement-fixing (IgG1) antibodies to MHC (Wasowska, 2010; and Murata et al. 2007). Thus, the CDC activity in the Donor 2 starting serum could have resulted from a combination of anti-HLA-DR11 IgG1 and complement-fixing antibodies without specificity for HLA-DR11, while the HLA-DR11 column eliminated HLA-DR11-elicited CDC activity from both the flow-through and eluate by separating these syngergistic antibodies. These results show that HLA immunoaffinity columns absorb complement-fixing activity in a sera-specific manner.

A long-term objective in the development of an HLA immunoaffinity matrix is antibody absorption. Antibody reduction therapies such as plasma exchange are now used for bulk antibody depletion to facilitate transplants for recipients who are otherwise serologically incompatible. One drawback to these existing reduction therapies is their lack of specificity, which results in the removal of beneficial as well as deleterious anti-HLA antibodies (Schmaldienst et al., 2001). The ability to specifically deplete anti-HLA antibodies could significantly improve existing immune reduction therapies. Antigen-specific antibody depletion columns are currently in use to remove antibodies specific for blood group A and B antigens (Crew et al., 2010; and Takahashi, 2007). While the column and serum volumes tested here were on a small scale, this column could be scaled up, similar to the columns for blood group antigens, in order to reduce anti-HLA-antibodies from patient plasma before or after transplantation.

In summary, an approach for producing milligram quantities of native class II HLA proteins in mammalian cells has been developed, and in this Example, these proteins have been successfully coupled to a column support used to purify anti-HLA antibodies. The DR11 reactive antibodies recovered were functionally intact and highly crossreactive. Antibodies that recognized DR11 fixed complement in one of the two donors, and isotype profiles were consistent with CDC activity. These observations demonstrate that HLA immunoaffinity columns, or perhaps other platforms such as HLA coated magnetic beads, will provide transplant physicians and their supporting clinical HLA laboratories with the means to parse anti-HLA reactivity into acceptable or unacceptable categories on the basis of CDC activity, isotype profile, and serologic cross-reactivity with other HLA. HLA technologies like this antibody separation device will help elucidate which antibodies promote rejection. Lastly, these results establish the feasibility of using HLA immunoaffinity columns to study anti-HLA immunity and to achieve specific immune reduction for organ transplantation.

### EXAMPLE 4

### SHARC (sHLA Antibody Removal Column) Analysis

Coupling CNBr (Cyanogen Bromide) vs NHS (N-Hydroxysuccinimide)

There are three primary properties of a matrix that indicate the effectiveness of the SHARC. These properties are: (1) coupling efficiency—the ability of an activated matrix to covalently link sHLA to the solid support; (2) binding capacity—the maximum quantity of antibody depleted by the sHLA linked matrix; and (3) regeneration efficiency—the number of times the matrix can be loaded and eluted (regenerated).

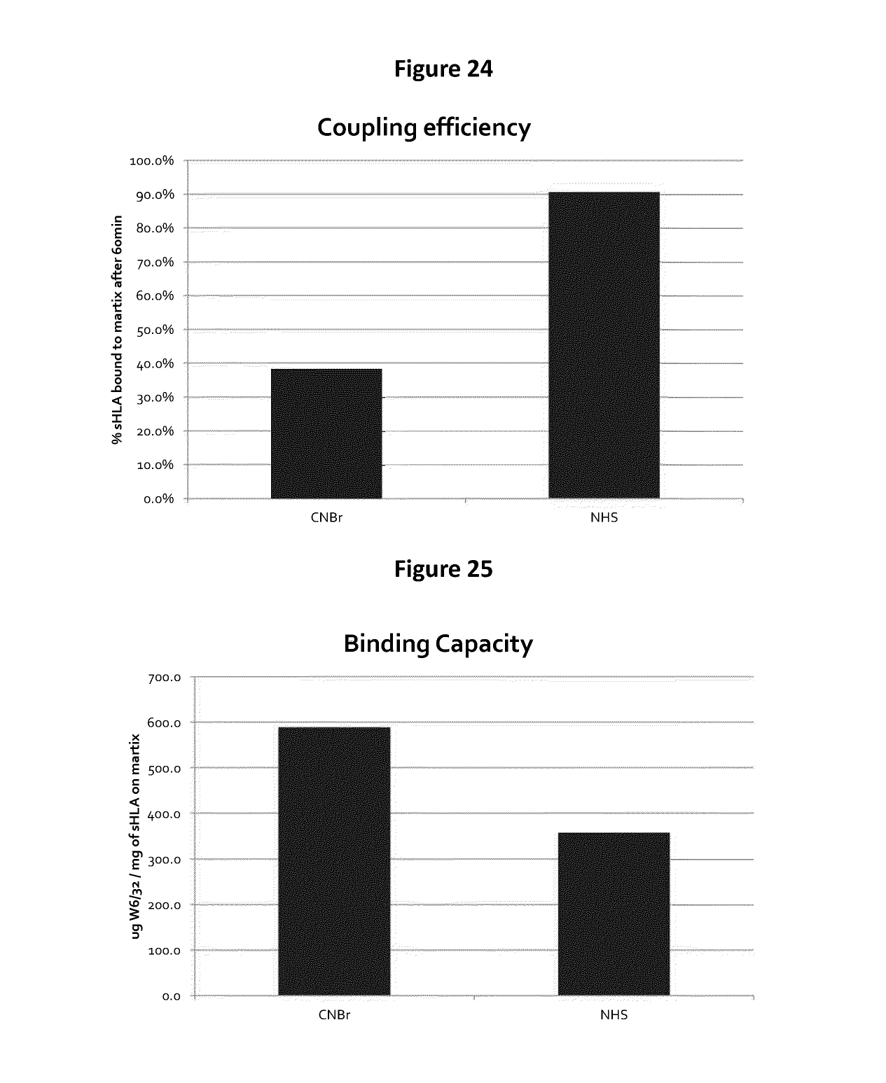

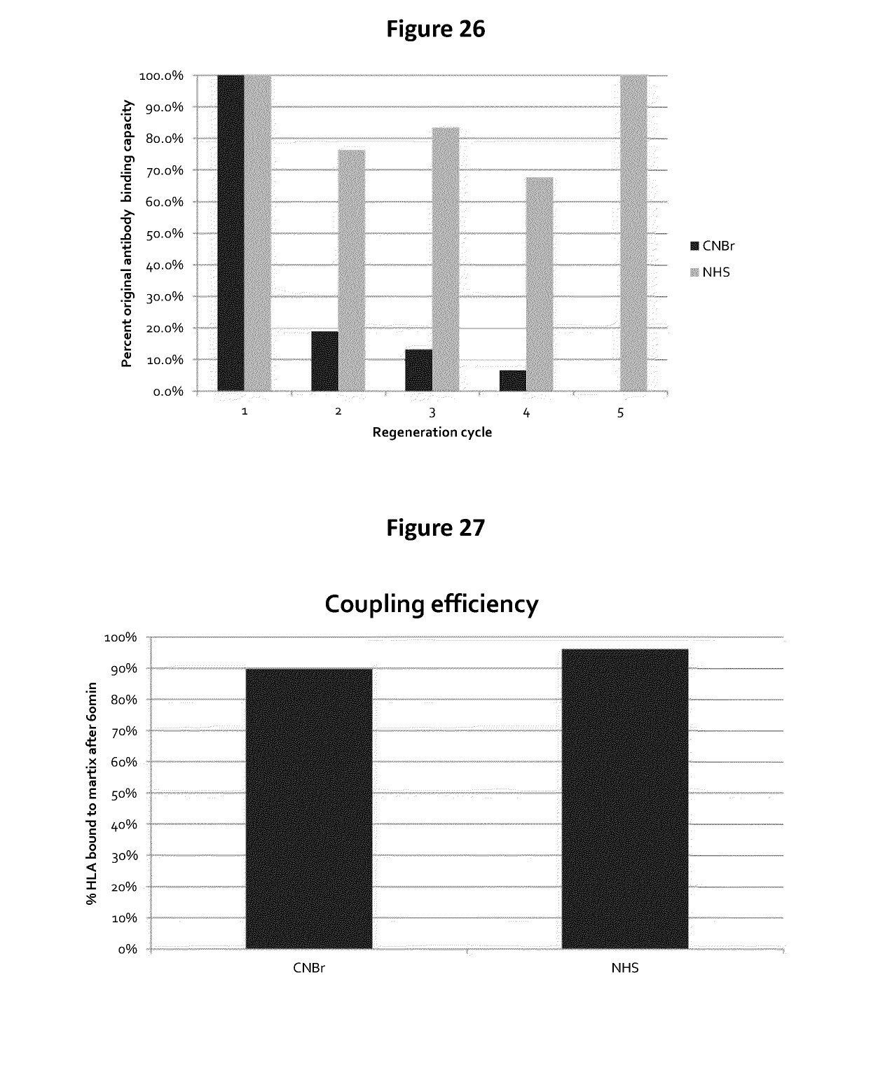

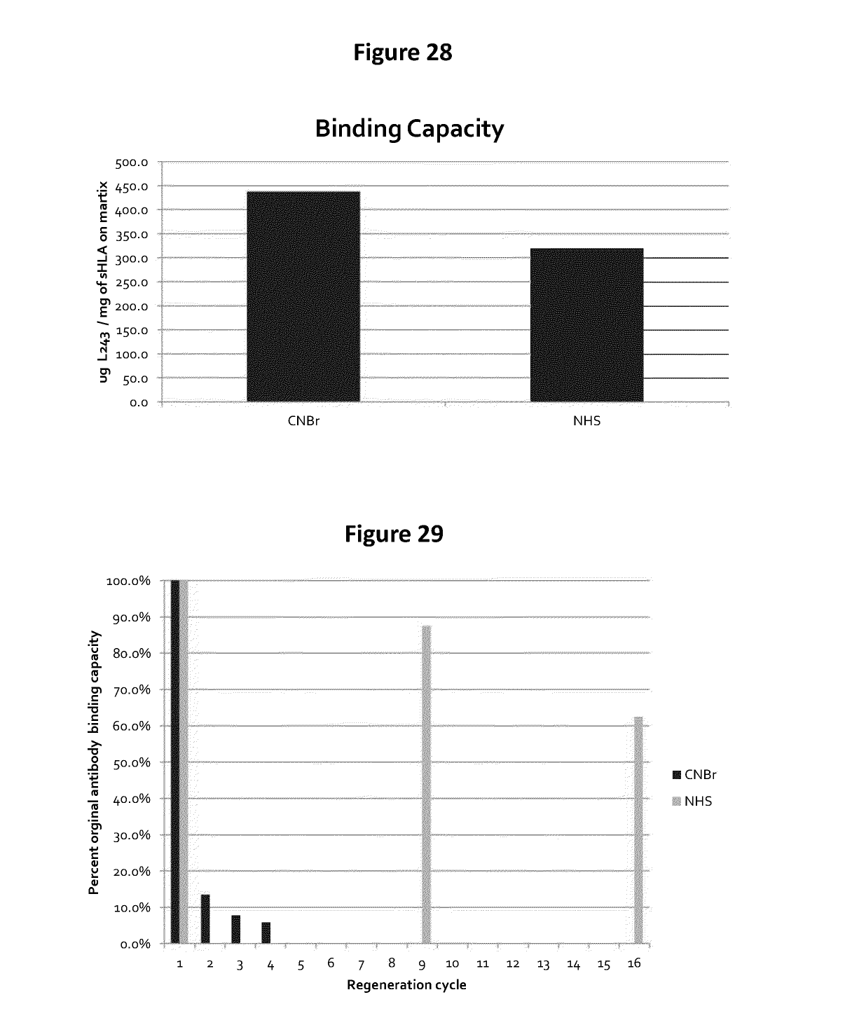

sHLA can be covalently linked to a solid support such as SEPHAROSE® using a number of different chemistries. In this Example, the aforementioned parameters were tested with either a CNBr- or NHS-SEPHAROSE® based chemistry to link sHLA to a SEPHAROSE® 4 fast flow matrix. Both CNBr and NHS chemistries were tested using class I and class II sHLA. In the case of class I sHLA, the NHS-based chemistry outperformed in both coupling efficiency (FIG. 24) and regeneration efficiency (FIG. 25); however, it exhibited a lower binding capacity (FIG. 25). For class II sHLA, coupling efficiencies were similar between NHS and CNBr (FIG. 27), but the binding capacity was higher with the CNBr matrix (FIG. 28); in addition, the regeneration efficiency was higher with the NHS matrix.

Full Scale Class I and Class II HLA SHARC

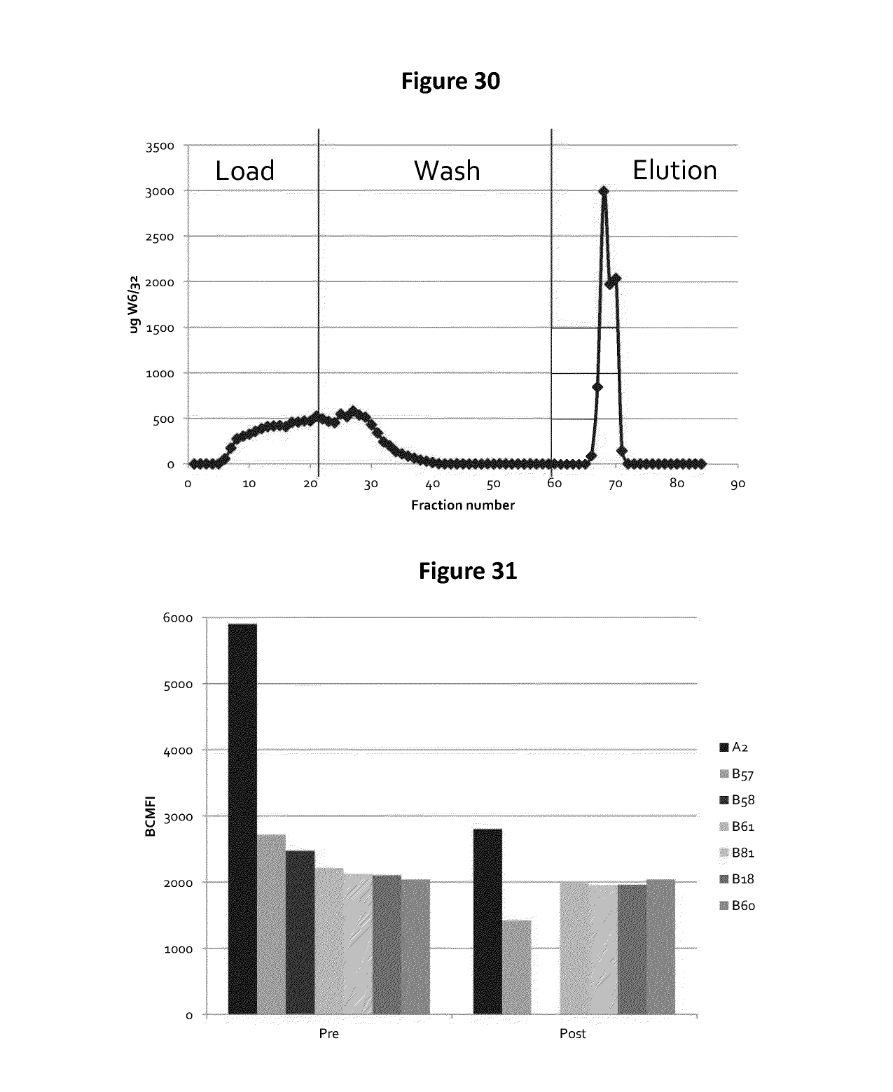

In order to demonstrate that the full scale SHARC was able to deplete anti-HLA antibodies, the ability to deplete monoclonal anti-HLA antibodies from PBS was first investigated. In these experiments, sHLA-A2 was used as the class I molecule, and sHLA-DR11 was used as the class II molecule. The pan-class I antibody W6/32 was used for analysis of class I, while the pan-HLA-DR antibody L243 was used for class II. As shown in FIGS. 30 and 33, both the sHLA-A2 (class I) and sHLA-DR11 (class II) SHARC devices depleted anti-HLA antibodies from PBS, although the sHLA-DR11 SHARC was more effective than the HLA-A2 SHARC.

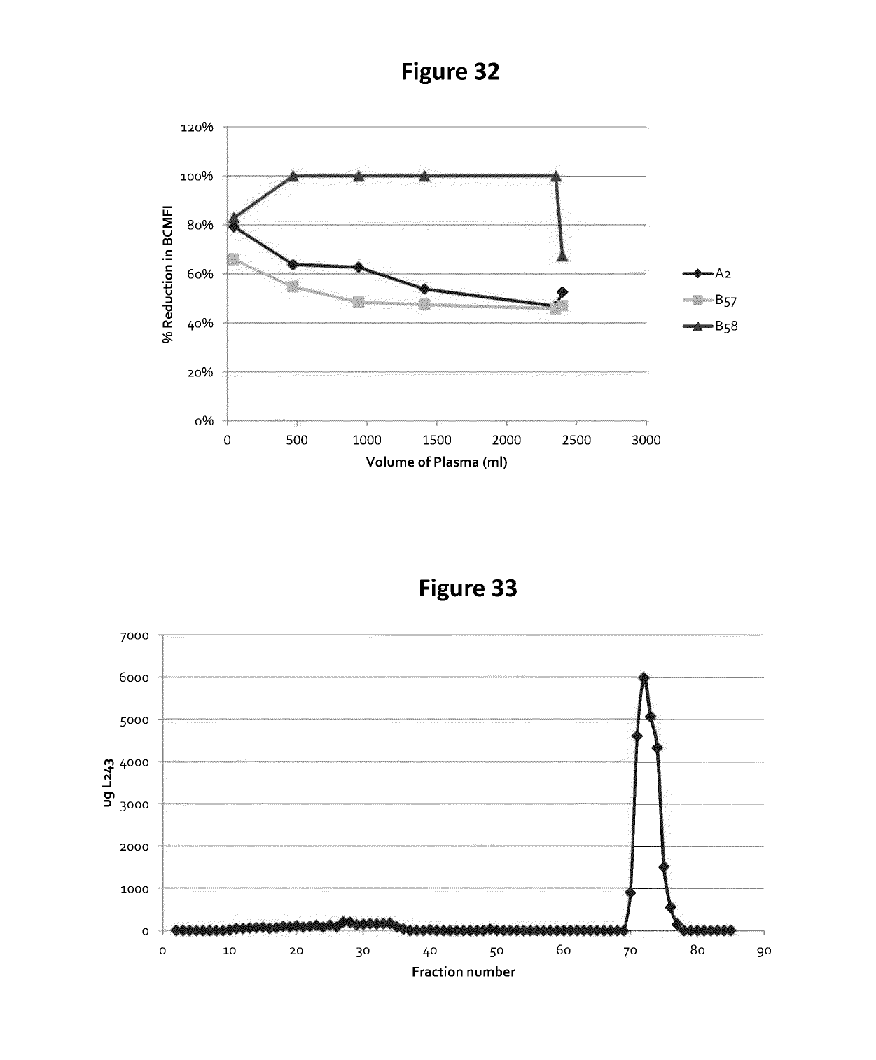

Next, the ability of the class I and II SHARC to deplete antibody from patient plasma containing anti-HLA antibodies was tested. When patient plasma containing anti-HLA-A2 antibodies was passed over the sHLA-A2 SHARC, anti-HLA-A2 antibodies were depleted (FIGS. 31 and 32). In addition to anti-HLA-A2 antibodies, serologically related antibodies (B57, B58) were reduced from the starting plasma. The presence of serologically unrelated anti-HLA antibodies (B61, B81, B18, B60) was unchanged between pre-SHARC and post-SHARC plasma, demonstrating that these antibodies passed through the SHARC without binding thereto (FIG. 31).

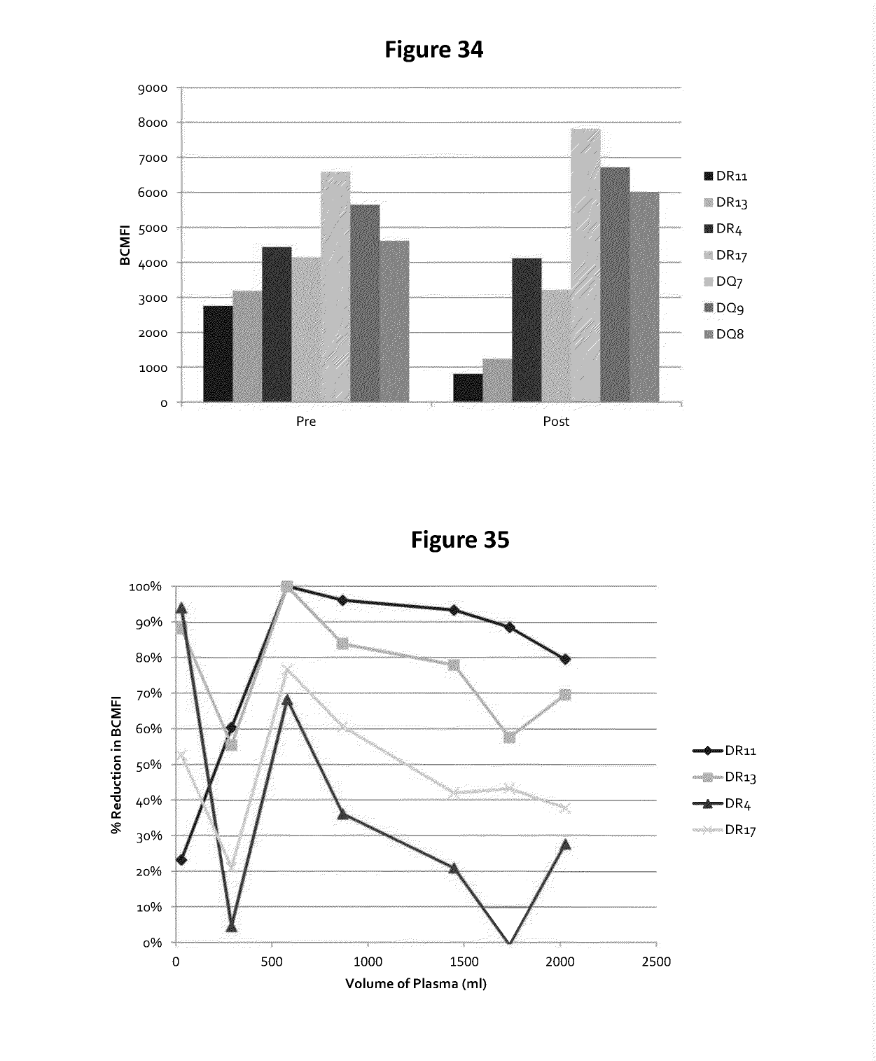

Like the sHLA-A2 SHARC, the sHLA-DR11 SHARC depleted anti-HLA-DR11 antibodies from patient plasma (FIGS. 34 and 35). As shown in FIG. 34, anti-HLA-DR11 antibodies as well as serologically related antibodies (DR13, DR4, DR17) were reduced from the starting plasma. Serologically unrelated anti-HLA antibodies (DQ7, DQ8, DQ9) were unchanged between pre-SHARC and post-SHARC plasma, demonstrating that these antibodies passed through the SHARC without binding thereto. Together these data demonstrate the ability and specificity of both of the class I and II SHARC devices.

### EXAMPLE 5

In this Example, several specific HLA-A*0201 columns were generated to demonstrate the feasibility of removing defined anti-HLA antibodies (anti-HLA-mAbs) from a buffered solution. Soluble class I HLA molecules were produced in a native conformation in mammalian cells, purified by affinity chromatography, coupled to a SEPHAROSE® matrix, and loaded into a column enclosure. The HLA on these columns were shown to maintain their structural integrity and function. Multiple passes of the antibody W6/32, which recognizes only intact HLA molecules, resulted in consistent and repeatable binding patterns. During the entire evaluation process, several parameters were identified determining capacity and efficiency. In conclusion, the anti-HLA antibody removal devices have been demonstrated herein to be highly efficient in selectively depleting anti-HLA-mAbs.

### Materials and Methods for Example 5

Recombinant techniques were used to create cell lines which express single HLA class I molecules (as described herein above). Eliminating the cytoplasmic and transmembrane regions of the molecule resulted in a soluble form of HLA (sHLA) which is secreted during production and easily purified by affinity chromatography. Large-scale production of sHLA proteins was performed using the CP-2500 CELL PHARM® system. Hollow fiber bioreactors are designed to produce up to 50 to 100 times more protein than a traditional static culture will yield. Affinity chromatography purification was applied to purify crude sHLA harvests, resulting in samples of >95% purity. All samples produced were individually controlled by a QC system. Mass spectroscopy demonstrated that soluble HLA proteins were purified so that contaminants are essentially undetectable.

After purification of sHLA, fractions of the protein stock were used to couple to NHS-activated SEPHAROSE® 4 Fast Flow and packed into a chromatography column. Elution profiling was conducted using an Akta Purifier System by applying a specific run protocol consisting of a loading cycle, elution cycle, and equilibration cycle. All parameters were kept consistent throughout the study, assigning a volume of 12 ml of PBS, pH 7.4 to the loading cycle, 8 ml of 0.1 M Glycine pH 11.0 to the elution cycle and 25 ml of PBS, pH 7.4 to equilibrate the system. Depending on the injection amount and volume, different loading loops were used. Data showed that injection conditions are concentration independent (not shown).

FIG. 36 shows a typical coupling timeline for binding of the sHLA to the SEPHAROSE® 4 Fast Flow matrix. A rapid decline of sHLA is visualized within the first 10 minutes and faded out after 30 minutes, where no additional sHLA is bound to the matrix. For this Example, three columns of 0.5, 1.0 and 2.0 mg per ml matrix were created with coupling efficiencies above 95%.

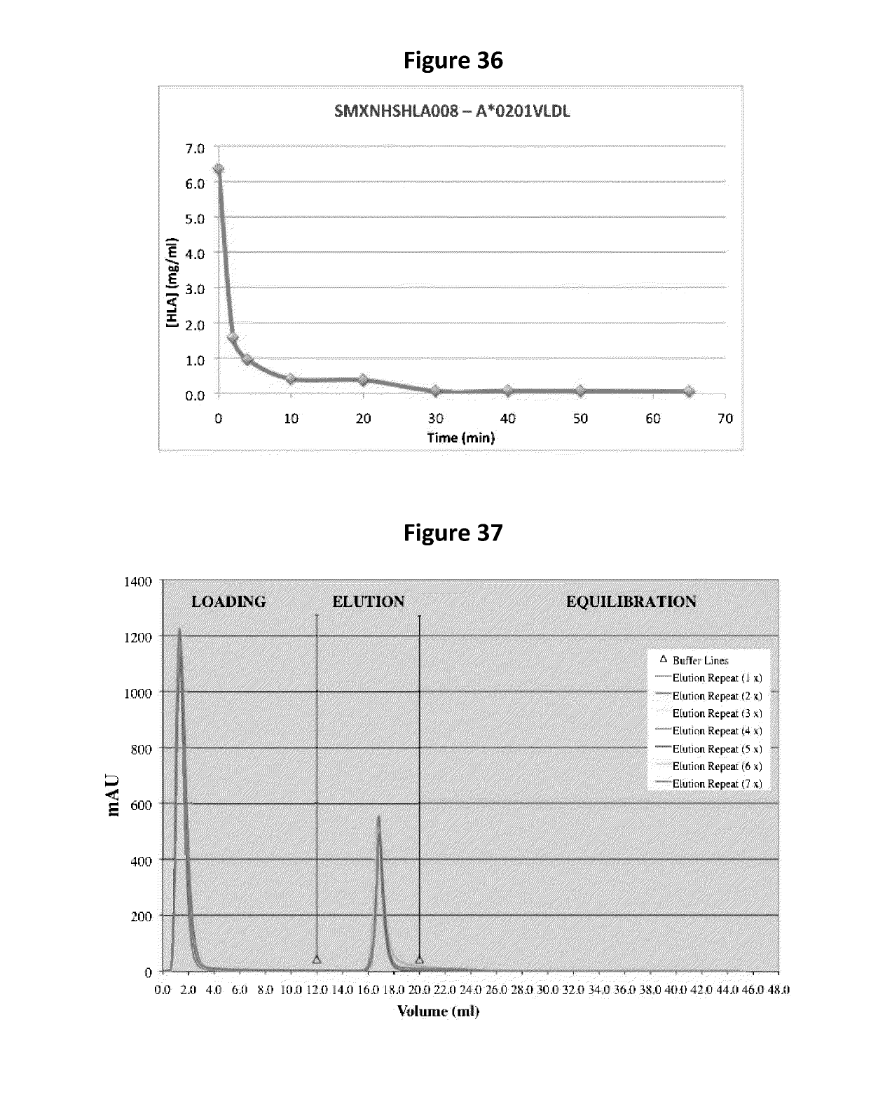

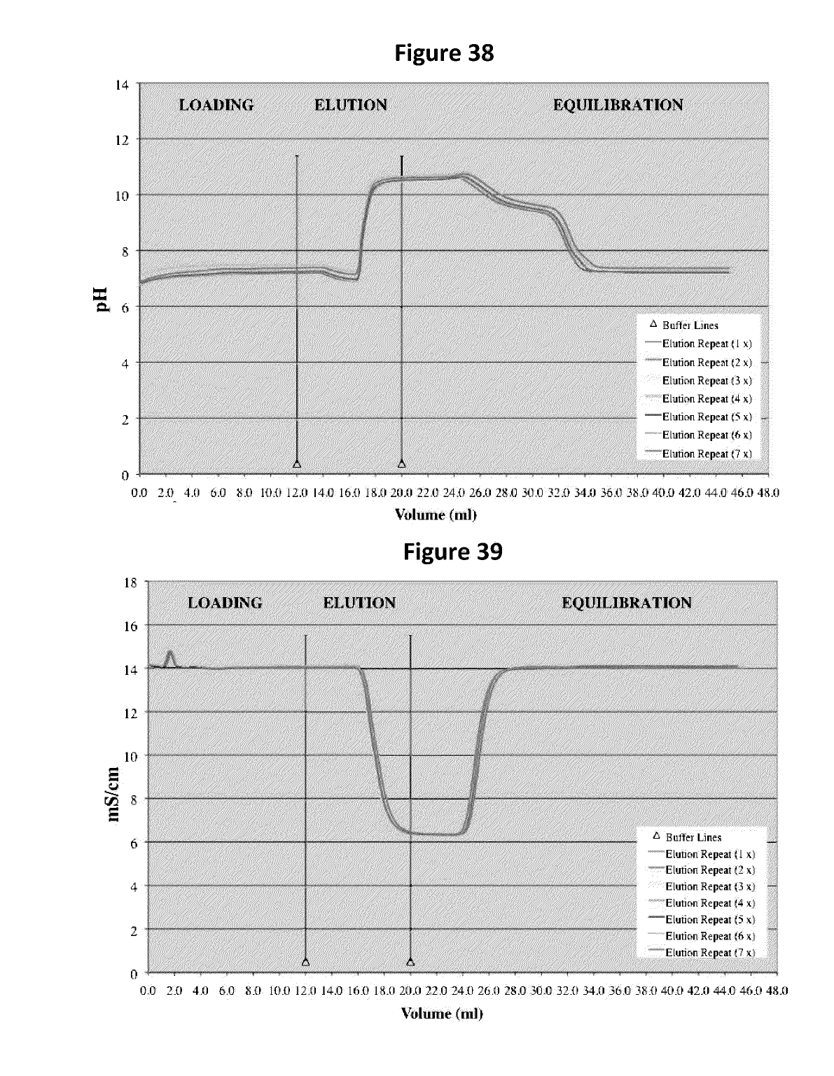

To assure consistency in the measurements, a repeatability study was started to record and superimpose elution profiles. For quality purposes, three parameters were observed: (1) Absorption Units (mAU) to detect proteinacious material (FIG. 37); (2) pH (FIG. 38); and (3) conductivity to follow changes in buffer phases (FIG. 39). The graphics prove great consistency between multiple experiments, validating the suitability of the method.

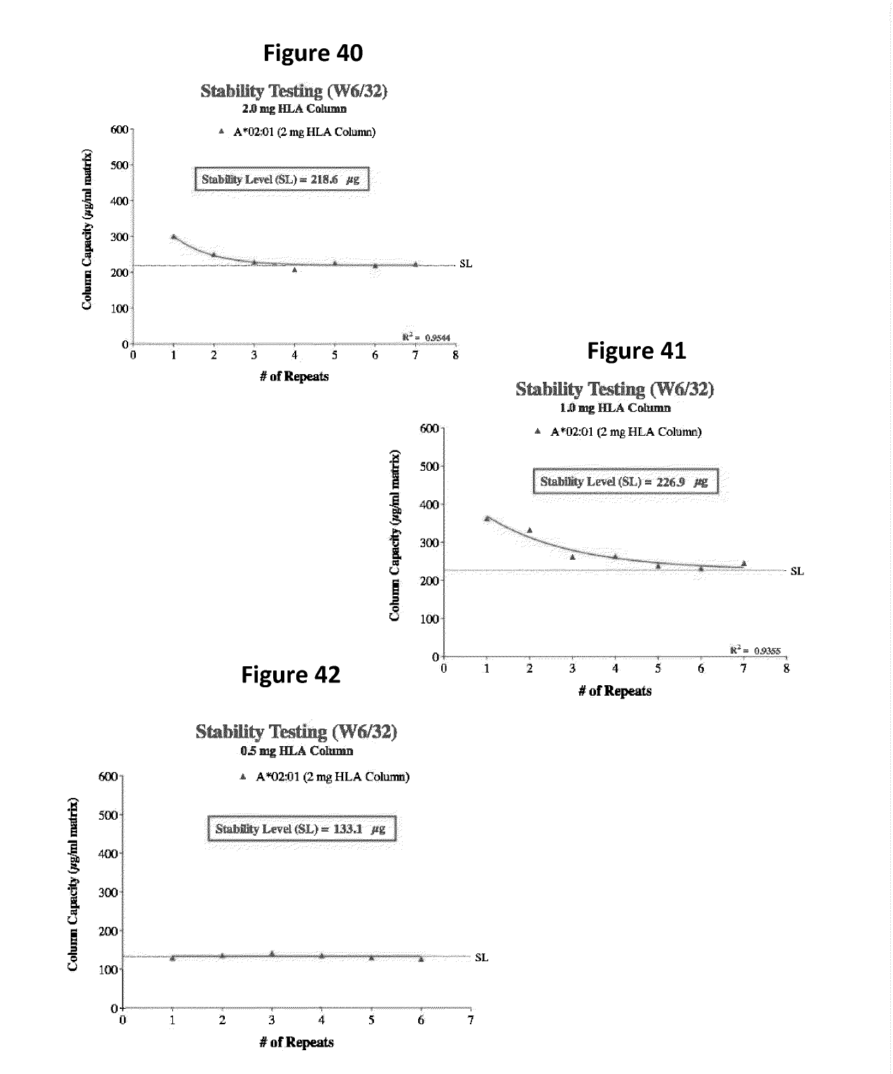

Using the anti-HLA-mAb W6/32, which recognizes only structurally intact HLA molecules, multiple rounds of load-elute-equilibrate cycles were performed to measure the stability of sHLA attached to the solid support (FIGS. 40-42). Overall it was observed that freshly coupled HLA-columns lose HLA molecules within the first 3 rounds of glycine exposure, but then stabilize with no further loss of functionality. This effect is most likely caused by incompletely coupled HLA proteins being trapped within the matrix and knocked loose after a drastic pH change. The effect seems to be more profound in higher coupling ratios. A similar study was performed manually (data not shown), measuring the “shedding” of sHLA after an elution event with the result that no sHLA was detectable after 5 elutions (15 cycles).

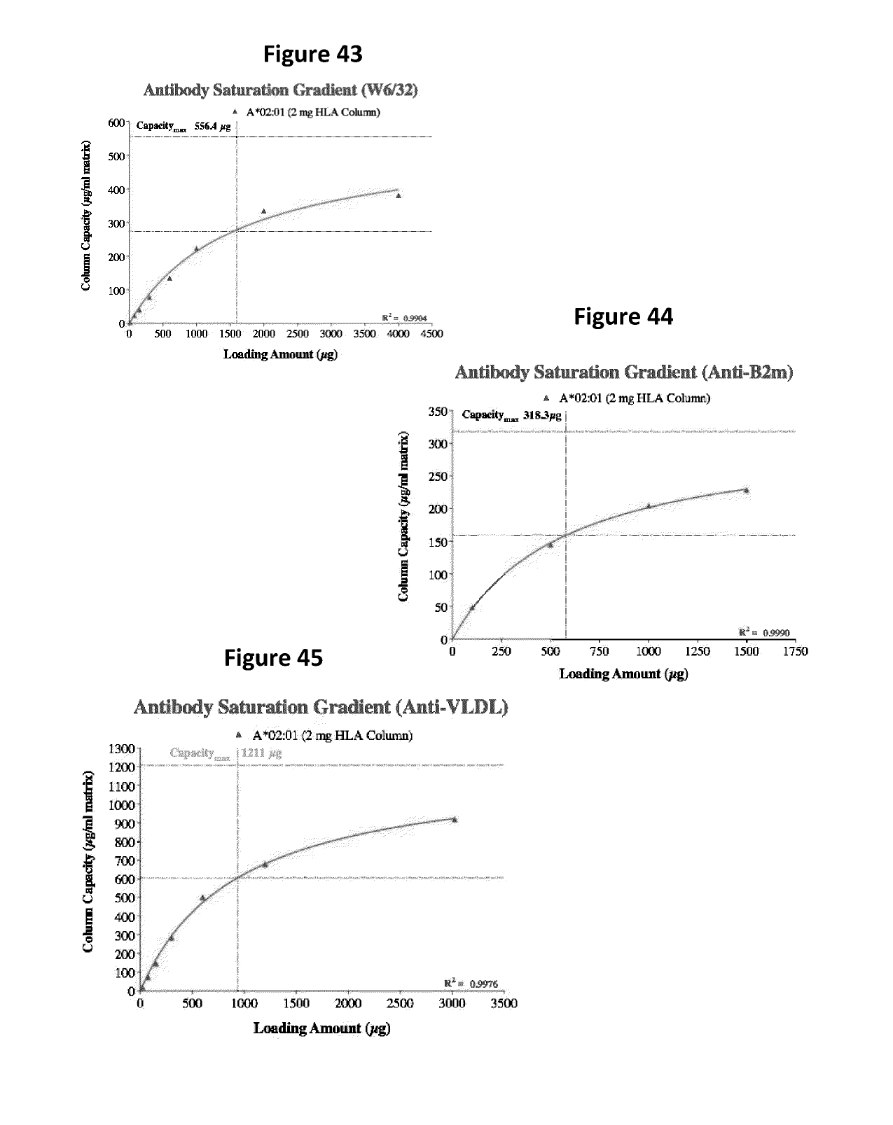

Determination of the column's binding capacity is one of the most important parameters in establishing feasibility of the technology. The more antibody that can be removed, the less sHLA is needed, and smaller/cheaper devices can be created. FIGS. 43-45 show three different anti-HLA mAbs applied to a 2.0 mg column at variable amounts. The column's capacity was shown to not be unlimited, but was able to bind a certain amount of antibodies before saturation occurred. Anti-VLDL (FIG. 45) appeared to be able to bind the largest amount of antibody before the column becomes saturated, while Anti-B2m (FIG. 44) bound the lowest amount of antibody before saturation. These differences were expected, as each antibody has a different affinity towards its target epitope. Depending on the anti-HLA mAb used, capacities ranged from 300-1200 μg of antibody per 1 ml matrix.

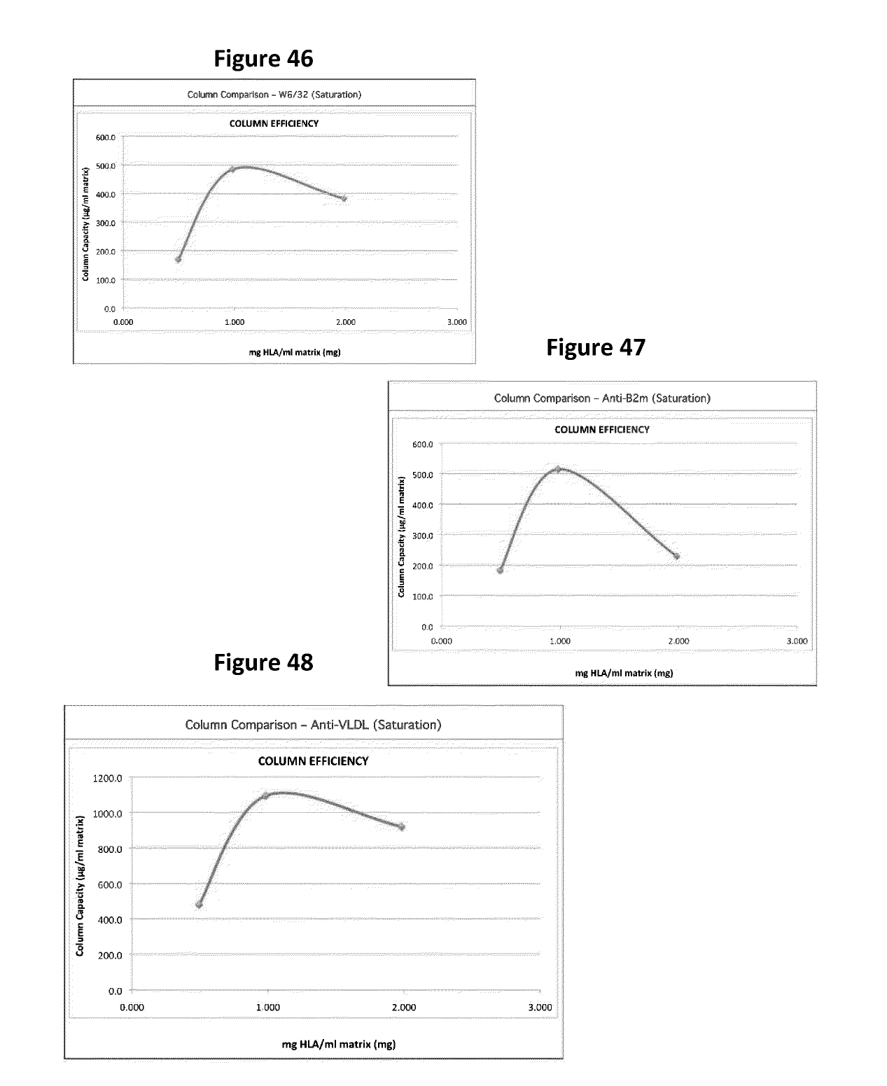

The maximum binding efficiency for the A*02:01 appeared to be at around 1 mg of HLA per 1 ml of matrix. This was confirmed by 3 independent tests using anti-HLA mAbs W6/32 (FIG. 46), anti-B2m (FIG. 47) and anti-VLDL (an antibody directed against an artificial tail introduced into the A*02:01 molecule; FIG. 48). Clear evidence of sterical hindrance was detectable, where the 1 mg column reached much higher binding capacity than its 2 mg counterpart.

This Example demonstrates that soluble HLA class I molecules coupled to an affinity matrix were capable of binding specific anti-HLA Abs. Elution profiles become stable and the column performed without a visual decrease in functionality. All parameters measured were highly acceptable to move forward in creating a large-scale device.

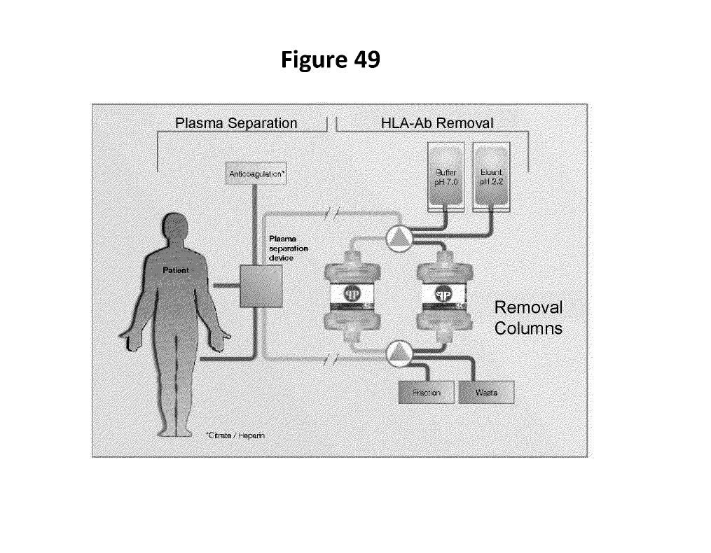

A proposed application scenario using such a system is shown in FIG. 49. The large amount of antibody required to be removed necessitates a two column system where one column is actively filtering plasma while the second is being regenerated.

Thus, in accordance with the presently disclosed and claimed inventive concept(s), there have been provided anti-MHC removal devices, as well as methods of production and use thereof, that fully satisfy the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference.
Adams, S. 2000. Nat Med 6:337-342.
Amico, P et al. 2009 (Epub Sep. 11, 2009). Curr Opin Organ Transplant. 14(6):656-61.
Bohmig, G. A., et al. 2000. Am J Kidney Dis 35:667-673.
Busch, et al. 2002 (Epub May 16, 2002). J Immunol Methods. 263(1-2):111-21.
Chang, H., et al. 1994. Immunology, 91:11408-11412.
Claas F H. 2010 (Epub Jul. 9, 2010). Curr Opin Organ Transplant. 15(4):462-6.
Clatworthy, M R et al. 2010 (Epub Oct. 12, 2010). Curr Opin Immunol. 22(5):669-81.
Crawford, F., et al. 1998. Immunity, 8:675-682.
Crew, R J et al. 2010 (Epub Jul. 9, 2010). Curr Opin Organ Transplant. 15(4):526-30.
Ditschkowski, M., et al. 1999. Ann Surg. 229(2): 246-254.
El-Awar, N et al. 2007 (Epub Jul. 22, 2008). Clin Transpl. 175-94.
Emonds, M. P., et al. 2000. Pediatr Transplant, 4:6-11.
Gaseitsiwe and Maeurer, 2009. Methods in Mol. Bio. 524:417-26.
Gloor, J et al. 2008 (Epub May 31, 2008). Am J Transplant. 8(7):1367-73.
Gronski and Weinem, 2006. Rev. Diabet. Stud. 3:88-95.
Guerra, C. B., et al. 1998. J. of Immunol., 160: 4289-4297.
Hansen, John A. 2005. Biology of Blood and Marrow Transplantation 11:24-27.
Herold et al., 2009. Clin Immunol. 132:166-173.
Hickman, H., et al. 2003. J. of Immunology, 171: 22-26.
Howden, A. J., et al. 2000. Hum Immunol, 61:419-24.
Jones et al., 2006. Nat. Rev. Immunol. 6:271-282.
Kalandadze, A et al. 1996 (Epub Aug. 16, 1996). J Biol Chem. 271(33):20156-62.
Kalandadze, A., et al. 1996. J. Bio. Chem. 271:20156-20162.
Kaufman and Herold. 2009. Diabetes Metab. Res. Rev. 25:302-6.
Kezuka, T., et al., 2001. Arch Ophthalmol. 119(7):1044-9.
Lampson, L. and Levy, R. 1980. J. Immunol. 125: 293-299.
Landschulz, W., et al. 1988. Science, Vol. 240.
McMurtrey, C., et al. 2008. PNAS, 105:2981-2986.
Muller-Steinhardt, M., et al. 2000. Clin Transplant, 14:85-9.
Murata, K et al. 2007 (Epub Sep. 18, 2007). Am J Transplant. 7(11):2605-14.
Nankivell, B J et al. 2010 (Epub Oct. 12, 2010). N Engl J Med. 363(15):1451-62.
Nepom and Kwok, 1998. Diabetes, 47:1177-84.
Novak, E., et al. 1999. J. Clin. Invest. 104:R63-R67.
Pratesi, F., et al. 2000. J Rheumatol, 27:109-15.
Prilliman, K. R., et al. 1999. J. Immunology, 162:7277.
Schmaldienst, S et al. 2001 (Epub May 24, 2001). Rheumatology (Oxford). 40(5):513-21.
Schuna, A. A. and C. Megeff. 2000. Am J Health Syst Pharm, 57:225-34. 2000.
Streilein, J W et al. 2007. Ocul Immunol Inflamm. 15(3):187-94.
Takahashi, K. 2007 (Epub Jun. 27, 2007). Clin Exp Nephrol. 11(2):128-41.
Todd et al., 1988. Science, 240:1003-1009.
Warren, D S et al. 2010 (Epub Jan. 21, 2010). Immunol Res. 47(1-3):257-64.
Wasowska, B A. 2010 (Epub Feb. 6, 2010). Immunol Res. 47(1-3):25-44.
Weber et al., 2007. Oncologist. 12:864-72.
Wettstein, D. A., et al. 1991. J. Exp. Med. 174:219-228.
Wicker et al., 1996. J. Clin. Invest. 98:2597-2603.
Wilson, B., 1981. Scand. J. Immunol., 14:201-205.
Yoon and Jun, 2001. Ann N Y Acad Sci. 928:200-11.
Yoshida and Kikutani, 2000. Rev. Immunogenet. 2:140-6.
Zimmer, K. P., et al. 1995. Gut. 36: 703-709.

What is claimed is:

1. An anti-MHC removal device, comprising:
a solid support;
a serologically active, soluble MHC moiety covalently coupled to the solid support and disposed on a surface of the anti-MHC removal device, the MHC moiety capable of interacting with a sample brought into contact with the surface of the device having the serologically active, soluble MHC moiety disposed thereon, whereby antibodies specific for the MHC moiety present in the sample will bind thereto, resulting in removal of said antibodies from the sample.

2. The anti-MHC removal device of claim 1, wherein the MHC moiety is further defined as a soluble class I HLA trimolecular complex.

3. The anti-MHC removal device of claim 2, wherein the soluble class I HLA trimolecular complex is produced by a method comprising the steps of:
providing a nucleotide segment encoding a desired individual class I MHC heavy chain that has the coding regions encoding the cytoplasmic and transmembrane domains of the desired individual class I MHC heavy chain allele removed such that the nucleotide segment encodes a truncated, soluble form of the desired individual class I MHC heavy chain molecule;
cloning the nucleotide segment into a mammalian expression vector, thereby forming a construct that encodes the desired individual soluble class I MHC heavy chain molecule;
transfecting a mammalian cell line with the construct to provide a mammalian cell line expressing a construct that encodes a recombinant, individual soluble class I MHC heavy chain molecule, wherein the mammalian cell line is able to naturally process proteins into peptide ligands for loading into antigen binding grooves of MHC molecules, and wherein the mammalian cell line expresses beta-2-microglobulin;
culturing the mammalian cell line under conditions which allow for expression of the recombinant individual soluble class I MHC heavy chain molecule from the construct, such conditions also allowing for endogenous loading of a peptide ligand into the antigen binding groove of each recombinant, individual soluble class I MHC heavy chain molecule and non-covalent association of native, endogenously produced beta-2-microglobulin to form the individual soluble class I MHC trimolecular complexes prior to secretion of the individual soluble class I MHC trimolecular complexes from the cell;
harvesting the soluble class I MHC trimolecular complexes from the culture while retaining the mammalian cell line in culture for production of additional soluble class I MHC trimolecular complexes; and
purifying the individual, soluble class I MHC trimolecular complexes substantially away from other proteins, wherein the individual soluble class I MHC trimolecular complexes maintain the physical, functional and antigenic integrity of the native class I MHC trimolecular complex, and wherein each trimolecular complex so purified comprises identical recombinant, individual soluble class I MHC heavy chain molecules.

4. The anti-MHC removal device of claim 1, wherein the MHC moiety is further defined as a soluble class II HLA trimolecular complex.

5. The anti-MHC removal device of claim 4, wherein the soluble class II HLA trimolecular complex is produced by a method comprising the steps of:
providing a first isolated nucleic acid segment encoding a soluble form of an alpha chain of a HLA class II molecule having a first domain of a super secondary structural motif attached thereto;
providing a second isolated nucleic acid segment encoding a soluble form of a beta chain of the HLA class II molecule having a second domain of the super secondary structural motif attached thereto;
inserting the first and second isolated nucleic acid segments into a mammalian cell line, wherein the mammalian cell line does not express endogenous HLA class II, and wherein the mammalian cell line comprises glycosylation mechanisms required for glycosylation of proteins produced therein and chaperone complexes required for peptide ligand loading into HLA class II molecules;
culturing the recombinant mammalian cell line under conditions that allow for expression of the soluble class II alpha and beta chains, association of the soluble class II alpha and beta chains through the first and second domains of the super secondary structural motif, glycosylation of the soluble class II alpha and beta chains, and loading of an antigen binding groove formed from the soluble class II alpha and beta chains with an endogenously produced, non-covalently associated peptide ligand, thereby producing soluble class II trimolecular complexes;
isolating the soluble class II trimolecular complexes secreted from the recombinant mammalian cell line; and
purifying the soluble class II trimolecular complexes substantially away from other proteins.

6. The anti-MHC removal device of claim 1, wherein the solid support is selected from the group consisting of a well, a bead, a membrane, a microtiter plate, a matrix, a pore, plastic, glass, a polymer, a polysaccharide, nylon, nitrocellulose, a paramagnetic compound, and combinations thereof.

7. The anti-MHC removal device of claim 6, wherein the solid support is further defined as a solid matrix.

8. The anti-MHC removal device of claim 7, wherein the solid support is further defined as an N-hydroxysuccinimide (NHS)-activated SEPHAROSE® matrix.

9. The anti-MHC removal device of claim 1, wherein the soluble MHC moiety is coupled to the solid support via a covalent amide bond formed between a primary amino group contained within the HLA moiety and an ester group contained in the solid support.

10. The anti-MHC removal device of claim 1, wherein the solid support further comprises a spacer arm.

11. The anti-MHC removal device of claim 1, further defined as a human use device.

12. The anti-MHC removal device of claim 11, further defined as an extracorporeal plasmapheresis human use device.

13. A kit containing the anti-MHC removal device of claim 1.

14. The kit of claim 13, further comprising at least one reagent for elution of antibodies from the anti-MHC removal device.

15. A method of removing anti-HLA antibodies from a biological sample, the method comprising the steps of:
(a) contacting a biological sample with the anti-MHC removal device of claim 1, whereby antibodies specific for the MHC moiety present on a surface of the anti-MHC removal device are removed from the biological sample; and
(b) recovering the biological sample, whereby the antibodies specific for the MHC moiety are substantially reduced in the recovered biological sample.

16. The method of claim 15 wherein the biological sample is selected from the group consisting of serum, tissue, blood, plasma, cerebrospinal fluid, tears, saliva, lymph, dialysis fluid, organ or tissue culture derived fluids, fluids extracted from physiological tissues, and combinations thereof.

17. The method of claim 15, further comprising repeating steps (a) and (b).

18. The method of claim 15, further comprising the step of eluting antibodies from the anti-MHC removal device.

19. The method of claim 15, wherein the MHC moiety is a class I MHC trimolecular complex.

20. The method of claim 15, wherein the MHC moiety is a class II MHC trimolecular complex.

21. The method of claim 15, wherein the solid support of the anti-MHC removal device is selected from the group consisting of a well, a bead, a membrane, a microtiter plate, a matrix, a pore, plastic, glass, a polymer, a polysaccharide, nylon, nitrocellulose, a paramagnetic compound, and combinations thereof.

22. The method of claim 21, wherein the solid support is further defined as an N-hydroxysuccinimide (NHS)-activated SEPHAROSE® matrix.

23. The method of claim 15, further comprising the step of placing the recovered biological sample back into the patient.
